SEQUENCE LISTING (2 sequences)

SEQ ID NO: 1 - DNA, 21 bp, Artificial sequence
  Forward PCR primer for UCP2
gctagcatggttggtttcaag

SEQ ID NO: 2 - DNA, 21 bp, Artificial sequence
  Reverse PCR primer for UCP2
gctagctcagaaaggtgaatc

(19) United States
(12) Patent Application Publication
(10) Pub. No.: US 2007/0092577 A1
(43) Pub. Date: Apr. 26, 2007

(54) Dietary calcium for reducing the production of reactive oxygen species

(75) Inventors: Michael B. Zemel, Knoxville, TN (US); Xiaocun Sun, Knoxville, TN (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Tennessee Research Foundation, Knoxville, TN (US)

(21) Appl. No.: 11/543,171

(22) Filed: Oct. 3, 2006

Related U.S. Application Data
(60) Provisional application No. 60/723,042, filed on Oct. 3, 2005.
(60) Provisional application No. 60/787,819, filed on Mar. 31, 2006.

(51) Int. Cl. A61K 35/20; A61K 33/06; C12Q 1/00

(52) U.S. Cl. 424/535; 435/4; 424/682

(57) ABSTRACT

The subject application provides a method of identifying or screening compounds or compositions suitable for reducing the production of reactive oxygen species (ROS) comprising: a) feeding (or orally administering) compositions comprising dietary material containing dietary calcium (or dietary calcium) to at least one subject; b) measuring intracellular concentrations of calcium in cells of said at least one subject, wherein a decrease of intracellular calcium concentration in said cells of said at least one test subject as compared to the intracellular concentrations of calcium in the cells of at least one control subject is indicative of a compound, composition, combination of compounds or combination of compositions suitable for use in reducing the production of ROS in a subject. Methods of treating ROS-related diseases comprising the oral administration of dietary material containing dietary calcium (or dietary calcium) are also provided.

### CROSS-REFERENCE TO RELATED APPLICATIONS

This application claims the benefit of U.S. Provisional Application Ser. No. 60/723,042, filed Oct. 3, 2005 and U.S. Provisional Application Ser. No. 60/787,819, filed Mar. 31, 2006.

### BACKGROUND OF THE INVENTION

Reactive oxygen species (ROS) production is increased in obesity and diabetes (Furukaw et al., 2004; Atabek et al., 2004; Lin et al., 2005; Sonta et al., 2004). It has been postulated that hyperglycemia and hyperlipidemia, key clinical manifestations of obesity and diabetes, may promote ROS production through multiple pathways (Inoguchi et al., 2000; Shangari et al., 2004; Chung et al., 2003). ROS are also associated with a variety of diseases or disorders. For example, ROS are associated with cataracts, heart disease, cancer, male infertility, aging, and various neurodegenerative diseases such as Alzheimer's disease, amyotrophic lateral sclerosis, Parkinson's disease, multiple sclerosis and aging.

Previous studies from have demonstrated an anti-obesity effect of dietary calcium, with increasing dietary calcium inhibiting lipogenesis, stimulating lipolysis and thermogenesis and increasing adipocyte apoptosis (Zemel, 2005a). These effects are mediated by suppression of 1α, 25-(OH)₂D₃-induced stimulation of Ca²⁺ influx and suppression of adipose UCP2 gene expression (Shi et al., 2001; Shi et al., 2002). Further, ROS production is modulated by mitochondrial uncoupling status and cytosol calcium signaling, and that 1α, 25(OH)₂D₃ regulates ROS production in cultured murine and human adipocytes (Sun et al., 2005).

### BRIEF DESCRIPTION OF THE INVENTION

The subject application provides a method of identifying or screening compounds or compositions suitable for reducing the production of reactive oxygen species (ROS) comprising: a) feeding (or orally administering) compositions comprising dietary material containing dietary calcium (or dietary calcium) to at least one subject; b) measuring intracellular concentrations of calcium in cells of said at least one subject, wherein a decrease of intracellular calcium concentration in said cells of said at least one test subject as compared to the intracellular concentrations of calcium in the cells of at least one control subject is indicative of a compound, composition, combination of compounds or combination of compositions suitable for use in reducing the production of ROS in a subject. Methods of treating ROS-related diseases comprising the oral administration of dietary material containing dietary calcium (or dietary calcium) are also provided.

### BRIEF DESCRIPTION OF THE FIGURES

FIG. 1: Adipose intracellular ROS production in wild-type and aP2-agouti transgenic mice. Values are presented as mean±SEM, n=6.

FIG. 2: Adipose NADPH oxidase expression in wild-type and aP2-agouti transgenic mice. Values are presented as mean±SEM, n=6.

FIG. 3: Effect of dietary calcium on body weight and fat pads weight in aP2-agouti transgenic mice. Values are presented as mean±SEM, n=10.

FIG. 4: Effect of dietary calcium on fasting blood glucose in aP2-agouti transgenic mice. Values are presented as mean±SEM, n=10. * indicates significant difference from the basal diet, p<0.05.

FIG. 5: Effect of dietary calcium on adipose intracellular ROS production in aP2-agouti transgenic mice. Values are presented as mean±SEM, n=10.

FIG. 6: Effect of dietary calcium on adipose NADPH oxidase expression in aP2-agouti transgenic mice. Values are presented as mean±SEM, n=10.

FIG. 7: Effect of dietary calcium on adipose intracellular calcium ([Ca²⁺]i) in aP2-agouti transgenic mice. Values are presented as mean±SEM, n=10.

FIG. 8: Effect of dietary calcium on adipose UCP2 expression in aP2-agouti transgenic mice. Values are presented as mean±SEM, n=10.

FIG. 9: Effect of dietary calcium on soleus muscle UCP3 expression in aP2-agouti transgenic mice. Values are presented as mean±SEM, n=10. * indicates significant difference from the basal diet, p<0.05.

FIG. 10: Effect of dietary calcium on soleus muscle NADPH oxidase in aP2-agouti transgenic mice. Values are presented as mean±SEM, n=10. * indicates significant difference from the basal diet, p<0.05.

FIG. 11: Effect of dietary calcium on adipose 11β-HSD expression in aP2-agouti transgenic mice. Values are presented as mean±SEM, n=10.

FIG. 12: Effect of H₂O₂ on DNA synthesis in cultured 3T3-L1 adipocytes. Adipocytes were treated with either H₂O₂ (100 nmol/L) or α-tocopherol(1 μmol/L), combined with or without GDP (100 μmol/L) or nifedipine (10 μmol/L) for 48 hours. Data are expressed as mean±SE (n=6). Different letters above the bars indicate a significant difference at level of p<0.05.

FIG. 13: ROS production in cultured 3T3-L1 adipocytes. Adipocytes were treated with either H₂O₂ (100 nmol/L) or α-tocopherol (1 μmol/L), combined with or without GDP (100 μmol/L) or nifedipine (10 μmol/L) for 48 hours. Data are expressed as mean±SE (n=6). Different letters above the bars indicate a significant difference at level of p<0.05.

FIG. 14: Mitochondrial potential in cultured wild-type 3T3-L1 adipocytes and UCP2 transfected 3T3-L1 adipocytes. Adipocytes were treated with either H₂O₂ (100 nmol/L) or α-tocopherol (1 μmol/L), combined with or without GDP (100 μmol/L) or nifedipine (10 μmol/L) for 48 hours. Data are expressed as mean±SE (n=6).

FIG. 15: Intracellular calcium ([Ca²⁺]i )in cultured 3T3-L1 adipocytes. Adipocytes were treated with either H₂O₂ (100 nmol/L) or H₂O₂ (100 nmol/L) plus α-tocopherol (1 μmol/L) for 4 hours. Data are expressed as mean±SE (n=6). Different letters above the bars indicate a significant difference at level of p<0.05.

FIG. 16: ROS production in cultured 3T3-L1 adipocytes. Adipocytes were treated with either glucose (30 mmol/L) or glucose (30 mmol/L) plus nifedipine (10 μmol/L), or glucose (30 mmol/L) plus GDP, or glucose (30 mmol/L) plus 1α, 25-(OH)₂D₃ for 48 hours. Data are expressed as mean±SE (n=6). Different letters above the bars indicate a significant difference at level of p<0.05.

FIG. 17: [Ca²⁺]i in cultured 3T3-L1 adipocytes. Adipocytes were treated with either glucose (30 mmol/L) or glucose (30 mmol/L) plus α-tocopherol (1 μmol/L) for 4 hours. Data are expressed as mean±SE (n=6). Different letters above the bars indicate a significant difference at level of p<0.05.

FIG. 18: Expression ratio of NADPH oxidase to 18s in cultured 3T3-L1 adipocytes. Adipocytes were treated with either glucose (30 mmol/L) or glucose (30 mmol/L ) plus nifedipine (10 μmol/L), glucose (30 mmol/L) plus GDP, or glucose (30 mmol/L) plus 1, 25-(OH)₂D₃ for 48 hours. Data are expressed as mean±SE (n=6). Different letters above the bars indicate a significant difference at level of p<0.05.

FIG. 19: Expression ratio of UCP2 to 18s in cultured 3T3-L1 adipocytes. Adipocytes were treated with either glucose (30 mmol/L) or glucose (30 mmol/L) plus nifedipine (10 μmol/L), glucose (30 mmol/L) plus GDP, or glucose (30 mmol/L) plus 1α, 25-(OH)₂D₃ for 48 hours. Data are expressed as mean±SE (n=6). Different letters above the bars indicate a significant difference at level of p<0.05.

FIG. 20: DNA synthesis in cultured 3T3-L1 adipocytes. Adipocytes were treated with either glucose (30 mmol/L) or glucose (30 mmol/L) plus nifedipine (10 μmol/L), glucose (30 mmol/L) plus GDP, or glucose (30 mmol/L) plus 1α, 25-(OH)₂D₃ for 48 hours. Data are expressed as mean±SE (n=6). Different letters above the bars indicate a significant difference at level of p<0.05.

FIG. 21: Expression ratio of cyclin A to 18s in cultured 3T3-L1 adipocytes. Adipocytes were treated with either glucose (30 mmol/L) or glucose (30 mmol/L) plus nifedipine (10 μmol/L), glucose (30 mmol/L) plus GDP, or glucose (30 mmol/L) plus 1α, 25-(OH)₂D₃ for 48 hours. Data are expressed as mean±SE (n=6). Different letters above the bars indicate a significant difference at level of p<0.05.

FIG. 22A shows adipose tissue TNFα expression ratio and FIG. 22B shows IL-6 expression ratio in aP2-agouti transgenic mice. Data are normalized to 18s expression. Values are presented as mean±SEM, n=6. Means with different letter differ, p<0.001.

FIG. 23A shows adipose tissue IL-15 expression, FIG. 23B shows Adipose adiponectin expression and FIG. 23C shows Muscle IL-15 expression in aP2-agouti transgenic mice. Data are normalized to 18s expression. Values are presented as mean±SEM, n=6. Means with different letter differ, p<0.03.

FIG. 24A shows TNFα expression and FIG. 24B shows IL-6 expression in differentiated 3T3-L1 adipocytes. Adipocytes were treatment with 10 nmol/L 1α, 25-(OH)₂-D₃, 10 μmol/L nifepipine, and 10 nmol/L 1α, 25-(OH)₂-D₃ plus 10 μmol/L nifepipine respectively for 48 h. Data are normalized to 18s expression. Values are presented as mean±SEM, n=6. Means with different letter differ, p<0.02. FIG. 24C illustrates plasma 1α, 25-(OH)₂-D₃ in aP2-agouti transgenic mice fed low calcium (basal) or high calcium diets. Values are presented as mean±SEM, n=10. Means with different letter differ, p=0.005.

FIG. 25A shows IL-6 expression, FIG. 25B shows IL-8 expression, FIG. 25C shows IL-15 expression and FIG. 25D shows adiponectin expression in differentiated Zen-bio human adipocytes. Adipocytes were treatment with 10 nmol/L 1α, 25-(OH)₂-D₃, 10 μmol/L nifepipine, and 10 nmol/L 1α, 25-(OH)₂-D₃ plus 10 μmol/L nifepipine respectively for 48 h. Data are normalized to 18s expression. Values are presented as mean±SEM, n=6. Means with different letter differ, p<0.005.

FIG. 26A shows Adiponectin expression and FIG. 26B shows IL-15 expression in differentiated 3T3-L1 adipocytes. Adipocytes were treatment with 100 nmol/L H₂O₂, 1 μmol/L α±tocopherol, and 100 nmol/L H₂O₂, 1 μmol/L α±tocopherol respectively for 48 h. Data are normalized to 18s expression. Values are presented as mean±SEM, n=6. Means with different letter differ, p<0.05. FIG. 26C: There was no direct effect of ROS on IL-15 expression; however, addition of α±tocopherol markedly increased IL-15 by 2.2-fold as compared to H₂O₂-treated cells (P=0.043).

FIG. 27 demonstrates that calcitriol increased MIF (FIG. 27A) and CD14 (FIG. 27B) expression in human adipocytes, and addition of a calcium channel antagonist (nifedipine) reversed this effect, indicating a role of intracellular calcium in mediating this effect.

FIG. 28 demonstrates that calcitriol increased MIF (FIG. 28A) and CD14 (FIG. 28B) expression in mouse (3T3-L1) adipocytes and the addition of a calcium channel antagonist (nifedipine) reversed this effect.

FIGS. 29, 30 and 31 show that calcitriol markedly stimulate inflammatory cytokines M-CSF (FIG. 29), MIP (FIG. 30) and IL-6 (FIG. 31) expression in 3T3-L1 adipocytes, and co-culture with RAW 264 macrophages enhance this effect, indicating a potential role of adipocytes in regulation of local resident macrophages activity and that calcitriol regulates this effect via a calcium and mitochondrial uncoupling-dependent mechanism. Main effects of chemical treatment and culture status were significant (p<0.02).

FIGS. 32A-D illustrate the effect of calcitriol on mouse cytokine protein production. Calcitriol markedly increases production of several cytokines in 3T3-L1 adipocytes, as indicated in the schematic diagram.

FIGS. 33A-D demonstrate that the effect of calcitriol on mouse cytokine protein production in a co-culture system. Calcitriol markedly increased cytokine production in a 3T3-L1 adipocytes-RAW264 macrophage co-culture, as indicated in the schematic diagram.

FIG. 34: MCP-1 expression in 3T3-L1 adipocytes.

FIGS. 35-36: Calcitriol stimulates expression of TNFα and IL-6. Calcitriol stimulated TNFα expression by 91% (FIG. 35) and IL-6 by 796% (FIG. 36) in RAW 264 macrophages cultured alone. These effects were blocked by adding nifedipine or DNP. Co-culture of macrophages with differentiated 3T3-L1 adipocytes markedly augmented TNFα (FIG. 35) and IL-6 (FIG. 36) expression in macrophages, and these effects were further enhanced by calcitriol.

FIG. 37: The high calcium diet was without effect on body weight, but the milk diet did induce a significant decrease in total body weight.

FIG. 38: Both the calcium and the milk diets caused significant decreases in body fat, with the milk diet eliciting a significantly greater effect.

FIG. 39: The milk group had significantly greater skeletal muscle mass than the calcium group (p=0.02) and a tendency towards greater skeletal muscle mass than the basal group (p=0.06).

FIG. 40: Liver weight was slightly, but significantly, reduced by the milk diet.

FIG. 41: The high calcium diet caused a reduction in plasma 1, 25-(OH)₂-D (calcitriol) (p=0.002), and there was a trend (p=0.059) towards a further decrease in plasma calcitriol on the high milk diet.

FIG. 42: Adipose tissue reactive oxygen species (ROS) production was significantly reduced by the high calcium diet (p=0.002) and further reduced by the milk diet (p=0.03).

FIG. 43: The high calcium diet caused a significant reduction in adipose tissue NADPH oxidase (Nox; one of the sources of intracellular ROS) expression (p=0.001) and there was a strong trend (p=0.056) towards a further suppression of NOX on the milk diet.

FIG. 44: Plasma MDA was significantly decreased by both the calcium and milk diets (p=0.001), with a significantly greater effect of the milk diet (p=0.039).

FIGS. 45-49: The high calcium diet resulted in suppression of inflammatory markers and an upregulation of anti-inflammatory markers, and the milk diet exerted a greater effect than the high calcium diet. Adipose tissue expression of TNF-α (FIG. 45), IL-6 (FIG. 46) and MCP (FIG. 47) were all significantly suppressed by the high calcium diet. Expression of each of these inflammatory cytokines was lower on the milk diet than on the high calcium diet, but this difference was only statistically evident as a trend for TNF-α (p=0.076). The calcium and milk diets caused significant reductions in the release of inflammatory cytokines (TNF-α, FIG. 48; IL6, FIG. 49) from adipose tissue. There was trend towards a greater effect of the milk vs. calcium diet, but this difference was not statistically significant.

FIGS. 50-51: The high calcium and milk diets increased adiponectin expression (p=0.001; FIG. 50) and IL-15 expression (p=0.001; FIG. 51), and there was a trend for a further increase on the milk diet vs. high calcium diet (p=0.073 for adiponectin; p=0.068 for IL-15).

FIG. 52: There was a marked increase in skeletal muscle IL-15 expression on the high calcium diet (p<0.001). I1-15 expression was further increased on the milk diet (p=0.07).

### DETAILED DESCRIPTION OF THE INVENTION

The subject application provides a method of screening compounds or compositions suitable for reducing the production of reactive oxygen species (ROS) comprising: a) contacting one or more adipocyte cell(s) with compositions comprising dietary material containing dietary calcium; b) measuring the intracellular concentrations of calcium in said adipocyte cell(s), wherein a decrease of intracellular calcium concentration in said adipocyte cell(s) is indicative of a compound or composition suitable for use in reducing the production of ROS. Cells suitable for these screening methods include 3T3-L1 adipocytes (ATCC, Manassas, Va.) and human adipocytes (Zen Bio, Inc., Research Triangle, N.C.). These cells can be maintained in culture as described in Example 2.

Another screening method provided by the subject application provides a method of identifying or screening compounds or compositions suitable for reducing the production of reactive oxygen species (ROS) comprising: a) feeding (or orally administering) compositions comprising dietary material containing dietary calcium (or dietary calcium) to at least one subject; b) measuring intracellular concentrations of calcium in cells of said at least one subject, wherein a decrease of intracellular calcium concentration in said cells of said at least one test subject as compared to the intracellular concentrations of calcium in the cells of at least one control subject is indicative of a compound, composition, combination of compounds or combination of compositions suitable for use in reducing the production of ROS in a subject. In some embodiments of the invention, intracellular concentrations of Ca²⁺ are measured in adipocyte cells (e.g., visceral adipocytes or cutaneous adipocytes).

As used herein, the term “subject” or “individual” includes mammals. Non-limiting examples of mammals include transgenic mice (such as aP2-agouti transgenic mice) or human test subjects. Other mammals include, and are not limited to, apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

“Dietary material containing dietary calcium” is defined herein as any item normally consumed in the diet of a human or mammal. Non-limiting examples of such dietary materials are salmon, beans, tofu, spinach, turnip greens, kale, broccoli, waffles, pancakes, pizza, milk, yogurt, cheeses, cottage cheese, ice cream, frozen yogurt, nutrient supplements, calcium fortified vitamin supplements, or liquids supplemented with calcium. Specifically excluded from such a definition are those compositions that would be prescribed by a physician or veterinarian for the treatment of a condition. Also specifically excluded from the definition of “dietary calcium” or “dietary material containing dietary calcium” are compounds found in compound libraries (such as chemical compound libraries or peptide libraries) and compositions comprising such compounds or peptides. Also excluded from the definition of “dietary material containing dietary calcium” is any source of calcium that does not form a part of the diet of a mammal or human.

The subject application also provides methods of treating diseases associated with reactive oxygen species (ROS) comprising the oral administration of dietary calcium or dietary material containing dietary calcium to an individual in need of such treatment in amounts sufficient to decrease the intracellular concentrations of calcium in the cells of the individual. In some embodiments, the methods of treating diseases associated with ROS also include a step that comprises the diagnosis or identification of an individual as having a disease or disorder associated with ROS or suffering from elevated ROS levels.

The subject application also provides methods of altering the expression of cytokines in an individual (or the cytokine profile of an individual) comprising the oral administration of dietary calcium or dietary material containing dietary calcium that decrease intracellular calcium levels to an individual in need of such treatment in amounts sufficient to decrease intracellular levels of calcium in the cells of the individual, decrease TNF-α, CD14, MIP, MIF, M-CSF, MCP-1, G-CSF or IL-6 expression (or any combination of the aforementioned cytokines) in the individual, and increase the expression of IL-15, adiponectin, or both IL-15 or adiponectin in the individual. Non-limiting examples of dietary calcium sources include dairy products, dietary supplements containing calcium, foodstuffs supplemented with calcium, or other foods high in calcium.

ROS associated diseases include, and are not limited to, cataracts, diabetes, Alzheimer's disease, heart disease, inflammation, cancer, male infertility, amyotrophic lateral sclerosis, Parkinson's disease, and multiple sclerosis and aging. Thus, the subject application provides methods of treating cataracts, Alzheimer's disease, heart disease, cancer, male infertility, amyotrophic lateral sclerosis, Parkinson's disease, and multiple sclerosis and aging that comprises the administration of compounds, compositions, combinations of compounds or combinations of compositions in amounts sufficient to decrease the intracellular levels of calcium in an individual.

As set forth herein, the subject application also provides the following non-limiting aspects of the invention:

A) An in vitro method of screening compounds or compositions suitable for reducing the production of reactive oxygen species (ROS) comprising:

a) contacting one or more cell(s) with a composition comprising dietary material containing dietary calcium (or dietary calcium); and

b) measuring one or more of the following parameters:
i) intracellular concentrations of calcium in said one or more cell(s), wherein a decrease of intracellular calcium concentration in said cell(s) is indicative of a compound or composition suitable for use in reducing the production of ROS;
ii) UCP2 expression in said one or more cell(s), wherein an increase in UCP2 expression in said cell(s) is indicative of a compound or composition suitable for use in reducing the production of ROS;
iii) NADPH oxidase expression in said one or more cell(s), wherein a decrease in NADPH oxidase expression in said cell(s) is indicative of a compound or composition suitable for use in reducing the production of ROS;
iv) UCP3 expression in said one or more cell(s), wherein an increase in UCP3 expression in said cell(s) is indicative of a compound or composition suitable for use in reducing the production of ROS;
v) NADPH oxidase expression in said one or more cell(s), wherein a decrease in NADPH oxidase expression in said cell(s) is indicative of a compound or composition suitable for use in reducing the production of ROS;
vi) 11β-HSD expression in said one or more cell(s), wherein a decrease in the expression of 11β-HSD in said cell(s) is indicative of a compound or composition suitable for use in reducing the production of ROS;
vii) TNF-α, CD14, MIF, M-CSF, MIP, MCP-1, G-CSF or IL-6 expression in said one or more cell(s), wherein a decrease in the expression of TNF-α, CD14, MIF (macrophage inhibitory factor), MIP (macrophage inhibitory protein), M-CSF (macrophage colony stimulating factor), G-CSF (granulocyte colony stimulating factor) or IL-6 in said cell(s) is indicative of a compound or composition suitable for use in reducing the production of ROS; or
viii) IL-15 or adiponectin expression in said one or more cell(s), wherein an increase in the expression of IL-15 or adiponectin in said cell(s) is indicative of a compound or composition suitable for use in reducing the production of ROS;

B) The embodiment as set forth in A, wherein said one or more cell(s) is a adipocyte or an adipocyte cell line;

C) An embodiment as set forth in A or B, wherein the one or more cell(s) is a/are human adipocyte(s) or a murine adipocyte;

D) An embodiment as set forth in A, B or C, wherein the one or more cell(s) are an adipocyte cell line;

E) An embodiment as set forth in A, B, C or D, wherein the one or more cell(s) are a human adipocyte cell line;

F) An embodiment as set forth in A, B, C or D, wherein the one or more cell(s) are a murine adipocyte cell line;

G) An embodiment as set forth in A, B or C, wherein the one or more cell(s) are a murine or human adipocyte;

H) An embodiment as set forth in G, wherein the murine or human adipocytes are obtained from visceral, or subcutaneous, or both visceral and subcutaneous fat tissue;

I) An embodiment as set forth in A, B, C, D, E, F, G, or H, wherein the cell(s) are obtained from a transgenic mouse;

J) An embodiment as set forth in I, wherein the transgenic mouse is an aP2-agouti transgenic mouse;

K) An embodiment as set forth in A, B, C, D, E, F or G, wherein the cell(s) 3T3-L1 adipocytes;

L) A method of identifying or screening compositions comprising dietary material containing dietary calcium suitable for reducing the production of reactive oxygen species (ROS) comprising:

a) orally administering compositions comprising dietary material containing dietary calcium to at least one test subject; and

b) measuring one or more of the following parameters:
i) intracellular calcium concentrations in cells of said at least one test subject and at least one control subject, wherein a decrease of intracellular calcium concentration in the cells of a test subject as compared to the intracellular concentrations of calcium in the cells of at least one control subject is indicative of a compound, composition, combination of compounds or combination of compositions suitable for use in reducing the production of ROS in a subject;
ii) UCP2 expression in cells of said at least one test subject and at least one control subject, wherein an increase of UCP2 expression in the cells of a test subject as compared to the UCP2 expression in the cells of at least one control subject is indicative of a compound, composition, combination of compounds or combination of compositions suitable for use in reducing the production of ROS in a subject;
iii) NADPH oxidase expression in cells of said at least one test subject and at least one control subject, wherein a decrease of NADPH oxidase expression in the cells of a test subject as compared to the NADPH oxidase expression in the cells of at least one control subject is indicative of a compound, composition, combination of compounds or combination of compositions suitable for use in reducing the production of ROS in a subject;
iv) UCP3 expression in skeletal muscle cells of said at least one test subject and at least one control subject, wherein an increase in UCP3 expression in the skeletal muscle cells of a test subject as compared to UCP3 expression in the skeletal muscle cells of at least one control subject is indicative of a compound, composition, combination of compounds or combination of compositions suitable for use in reducing the production of ROS in a subject;
v) NADPH oxidase expression in skeletal muscle cells of said at least one test subject and at least one control subject, wherein a decrease of NADPH oxidase expression in the skeletal muscle cells of a test subject as compared to the NADPH oxidase expression in the skeletal muscle cells of at least one control subject is indicative of a compound, composition, combination of compounds or combination of compositions suitable for use in reducing the production of ROS in a subject;
vi) 11β-HSD expression in visceral adipocyte tissue or cells of said at least one test subject and at least one control subject, wherein a decrease of 11β-HSD expression in the visceral adipocyte tissue or cells of a test subject as compared to the 11β-HSD expression in the visceral adipocyte tissue or cells of at least one control subject is indicative of a compound, composition, combination of compounds or combination of compositions suitable for use in reducing the production of ROS in a subject;
vii) TNF-α, CD14, MIF, MIP, M-CSF, MCP-1, G-CSF or IL-6 expression in said one or more cell(s), wherein a decrease in the expression of TNF-α, CD14, MIF, MIP, M-CSF, G-CSF or IL-6 in said cell(s) is indicative of a compound or composition suitable for use in reducing the production of ROS; or
viii) IL-15 or adiponectin expression in said one or more cell(s), wherein an increase in the expression of IL-15 or adiponectin in said cell(s) is indicative of a compound or composition suitable for use in reducing the production of ROS;

M) An embodiment as set forth in L(b)(i)-(iii), (vii), or (viii), wherein the cells are adipocyte cells obtained from at least one test subject and at least one control subject;

N) An embodiment as set forth in M, wherein the cells are cutaneous adipocyte cells obtained from at least one test subject and at least one control subject;

O) An embodiment as set forth in M, wherein the intracellular concentration of calcium is measured in visceral adipocyte cells obtained from at least one test subject and at least one control subject;

P) An embodiment as set forth in M, wherein the intracellular concentration of calcium is measured in cutaneous, or visceral, or both cutaneous and visceral adipocyte cells obtained from at least one test subject and at least one control subject;

Q) An embodiment as set forth in L, M, N, O or P, wherein the test subject and control subject are human;

R) An embodiment as set forth in L, M, N, O or P, wherein the test subject and control subject are murine;

S) An embodiment as set forth in R, wherein the test subject and control subject are transgenic mice;

T) An embodiment as set forth in S, wherein the test subject and control subject are aP2-agouti transgenic mice;

Levels of NADPH oxidase, UCP2, UCP3, cyclin A, 11β-HSD, TNF-α, CD14, MIF, MIP, M-CSF, G-CSF, IL-6, IL-15, adiponectin and/or intracellular levels of calcium can be measured according to methods well-known in the art or as set forth in the following examples. By way of non-limiting examples, relative levels of expressions of NADPH oxidase, UCP2, UCP3, cyclin A, 11β-HSD, TNF-α, CD 14, MIF, MIP, M-CSF, G-CSF, IL-6, IL-15, and/or adiponectin can be determined by: 1) nuclear run-on assay, 2) slot blot assay, 3) Northern blot assay (Alwine et al., 1977), 4) magnetic particle separation, 5) nucleic acid or DNA chips, 6) reverse Northern blot assay, 7) dot blot assay, 8) in situ hybridization, 9) RNase protection assay (Melton et al., 1984, and as described in the 1998 catalog of Ambion, Inc., Austin, Tex.), 10) ligase chain reaction, 11) polymerase chain reaction (PCR), 12) reverse transcriptase (RT)-PCR (Berchtold et al., 1989), 13) differential display RT-PCR (DDRT-PCR) or other suitable combinations of techniques and assays. Labels suitable for use in these detection methodologies include, and are not limited to 1) radioactive labels, 2) enzyme labels, 3) chemiluminescent labels, 4) fluorescent labels, 5) magnetic labels, or other suitable labels, including those set forth below. These methodologies and labels are well known in the art and widely available to the skilled artisan. Likewise, methods of incorporating labels into the nucleic acids are also well known to the skilled artisan.

Alternatively, the expression of NADPH oxidase, UCP2, UCP3, cyclin A, 11β-HSD, TNF-α, CD 14, MIF, MIP, M-CSF, G-CSF, IL-6, IL-15, and/or adiponectin can be measured at the polypeptide level by using labeled antibodies that specifically bind to the polypeptides in immunoassays such as commercially available protein arrays/assays, ELISA assays, RIA assays, Western blots or immunohistochemical assays. Reagents for such detection and/or quantification assays can be obtained from commercial sources or made by the skilled artisan according to methods well known in the art.

### EXAMPLE 1

### In Vivo Studies

Animals and Diets:

A. Animal Pilot Study

Six-week old male aP2-agouti transgenic mice and wild-type male littermates (n=12/group) from our colony were utilized. Six mice randomly selected from each group were sacrificed to provide baseline data and the remaining 6 mice in each group were put on a modified AIN 93 G diet (Reeves 1997) with sucrose as the sole carbohydrate source and providing 64% of energy, and fat increased to 25% of energy with lard as previously described (Zemel et al., 2000; Sun et al., 2004). Mice were studied for 9 days, during which food intake and spillage were measured daily and body weight, fasting blood glucose, food consumption assessed weekly. At the conclusion of the study, all mice were killed under isofluorane anesthesia and fat pads were immediately excised, weighed and used for further study, as described below.

B. Diet Study

At 6 wk of age, 20 male aP2-agouti transgenic mice from our colony were randomly divided into two groups (10 mice/group) and fed a modified AIN 93 G diet with suboptimal calcium (calcium carbonate, 0.4%) or high calcium (calcium carbonate, 1.2%) respectively, with sucrose as the sole carbohydrate source and providing 64% of energy, and fat increased to 25% of energy with lard. Mice were studied for three weeks, during which food intake and spillage were measured daily and body weight, fasting blood glucose, food consumption assessed weekly. At the conclusion of the study, all mice were killed under isofluorane anesthesia and blood collected via cardiac puncture; fat pads and soleus muscle were immediately excised, weighed and used for further study, as described below.

This study was approved from an ethical standpoint by the Institutional Care and Use Committee of The University of Tennessee.

Measurement of Adipocyte Intracellular Ca²⁺ ([Ca²⁺]i)

Adipose tissue was first washed several times with Hank's Balanced Salt Solution (HBSS), minced into small pieces, and digested with 0.8 mg/ml type I collagenase in a shaking water bath at 37° C. for 30 min. Adipocytes were then filtered through sterile 500-μm nylon mesh and cultured in Dulbecco's Modified Eagle's Medium (DMEM) supplemented with 1% fetal bovine serum (FBS). Cells were cultured in suspension and maintained in a thin layer at the top of culture media for 2 h for cell recovery. [Ca²⁺]i in isolated mouse adipocytes was measured by using a fura-2 dual wavelength fluorescence imaging system. Prior to [Ca²⁺]i measurement, adipocytes were pre-incubated in serum-free medium for 2 h and rinsed with HBSS containing the following components (in mmol/L): NaCl 138, CaCl₂ 1.8, MgSO₄ 0.8, NaH₂PO₄ 0.9, NaHCO₃ 4, glucose 5, glutamine 6, Hepes 20, and bovine serum albumin 1%. Adipocytes were loaded with fura-2 acetoxymethyl ester (fura-2 AM) (10 μmol/L) in the same buffer in dark for 1 h at 37° C. Adipocytes were rinsed with HBSS three times to remove extracellular dye and then post-incubated at room temperature for an additional 30 min to permit complete hydrolysis of cytoplasmic fura-2 AM. A thin layer of adipocytes was plated in 35 mm dishes with glass cover slips (P35G-0-14-C, MatTek Corporation, Ashland, Mass.). The dishes with dye-loaded cells were mounted on the stage of Nikon TMS-F fluorescence inverted microscope with a Cohu 4915 CCD camera. Fluorescent images were captured alternatively at excitation wavelength of 340 nm and 380 nm with an emission wavelength of 520 nm. [Ca²⁺]i was calculated by using a ratio equation as described previously (Zemel, 2003).

Total RNA Extraction.

A total cellular RNA isolation kit (Ambion, Austin, Tex.) was used to extract total RNA from cells according to manufacturer's instruction.

Quantitative Real Time PCR

Adipocyte 18s, UCP2, NADPH oxidase and 11β-HSD, and muscle UCP3 and NADPH oxidase were quantitatively measured using a Smart Cycler Real Time PCR System (Cepheid, Sunnyvale, CA) with a TaqMan 1000 Core Reagent Kit (Applied Biosystems, Branchburg, N.J.). The primers and probe sets were obtained from Applied Biosystems TaqMan® Assays-on-Demand™ Gene Expression primers and probe set collection according to manufacture's instruction. Pooled adipocyte total RNA was serial-diluted in the range of 1.5625-25 ng and used to establish a standard curve; total RNAs for unknown samples were also diluted in this range. Reactions of quantitative RT-PCR for standards and unknown samples were also performed according to the instructions of Smart Cycler System (Cepheid, Sunnyvale, Calif.) and TaqMan Real Time PCR Core Kit (Applied Biosystems, Branchburg, N.J.). The mRNA quantitation for each sample was further normalized using the corresponding 18s quantitation (Sun et al., 2004c).

Determination of Intracellular ROS Generation

Adipose tissue digestion and adipocytes preparation were prepared as described in [Ca²⁺]i measurement. Intracellular ROS generation was assessed using 6-carboxy-2′, 7′-dichlorodihydrofluorescein diacetate (H2-DCFDA) as described previously (Manea et al., 2004). Cells were loaded with H2-DCFDA (2 μmol/L) 30 min before the end of the incubation period (48 h). After washing twice with PBS, cells were scraped and disrupted by sonication on ice (20 s). Fluorescence (emission 543 nm or 527 nm) and DNA content were then measured as described previously. The intensity of fluorescence was expressed as arbitary units per ng DNA.

Statistical Analysis.

Data were evaluated for statistical significance by analysis of variance (ANOVA), and significantly different group means were then separated by the least significant difference test by using SPSS (SPSS Inc, Chicago, Ill.). All data presented are expressed as mean±SEM.

Results

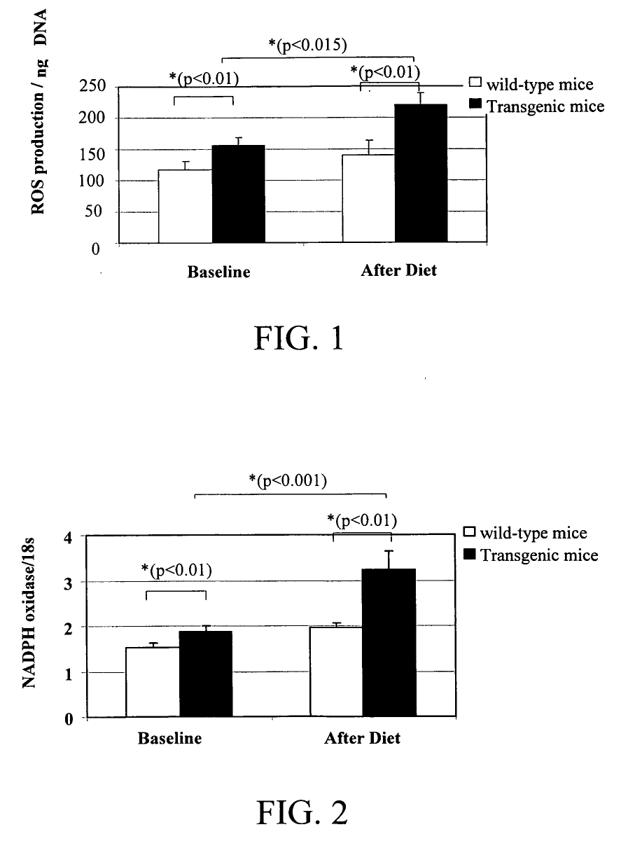

Our previous work indicated that aP2-agouti transgenic mice are a useful model for diet-induced obesity in a genetically susceptible human population, as they are non-obese on standard diets but develop mild to moderate obesity, hyperglycemia and insulin resistance when fed high sucrose and/or high fat diets (Zemel et al., 2000; Sun et al., 2004). Given the role of obesity and diabetes in oxidative stress, we first investigated whether aP2-agouti transgenic mice are also a suitable model for the study of diet-induced oxidative stress. Transgenic mice exhibited significantly greater baseline ROS production compared with wild-type controls prior to the feeding period, and the consumption of the obesity-promoting diet significantly increased adipose tissue ROS production only in aP2-agouti transgenic mice (FIG. 1). This effect was also associated with increased NADPH oxidase expression in adipose tissue of aP2-agouti transgenic mice prior to and following consumption of the obesity-promoting diet (FIG. 2).

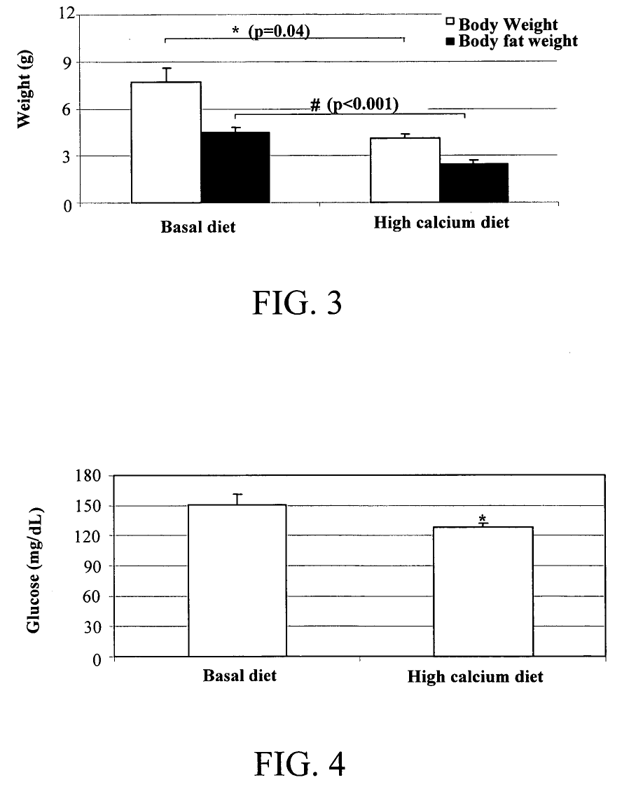

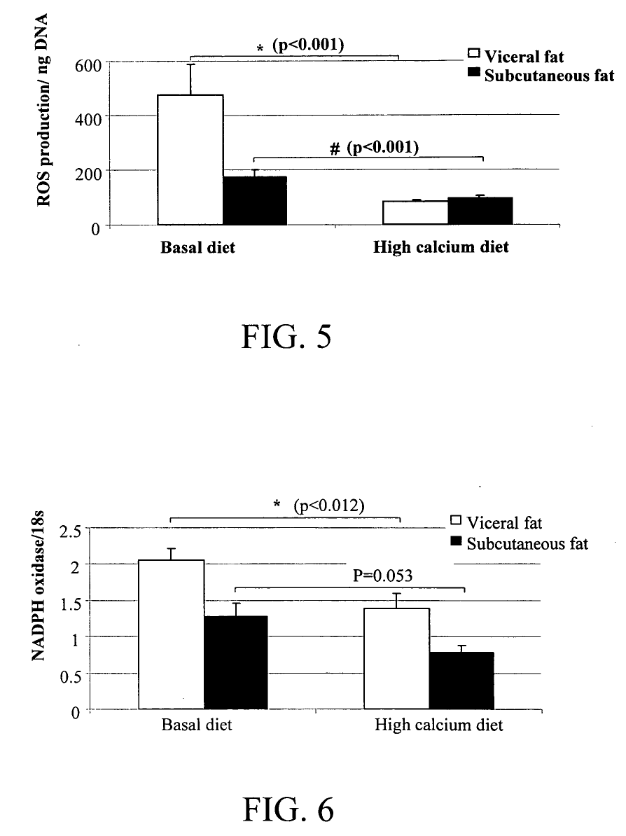

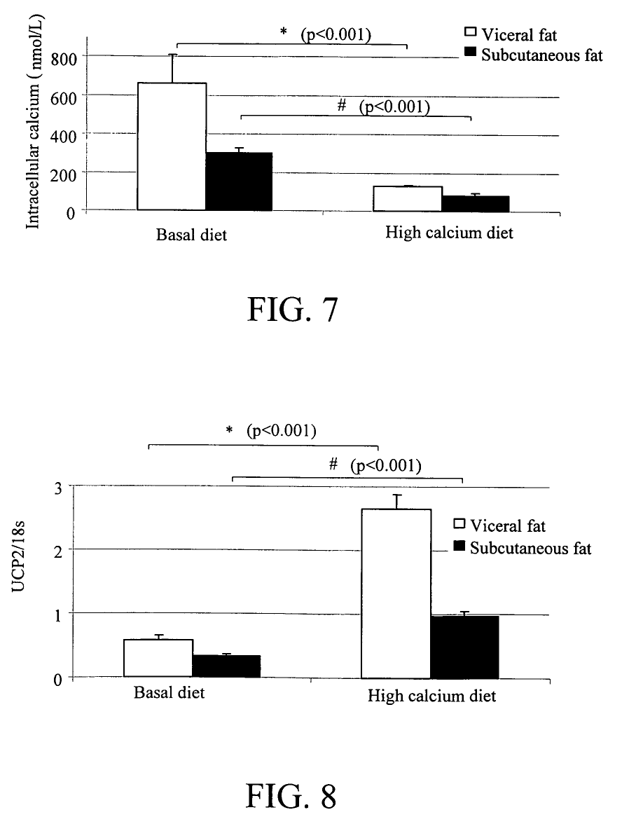

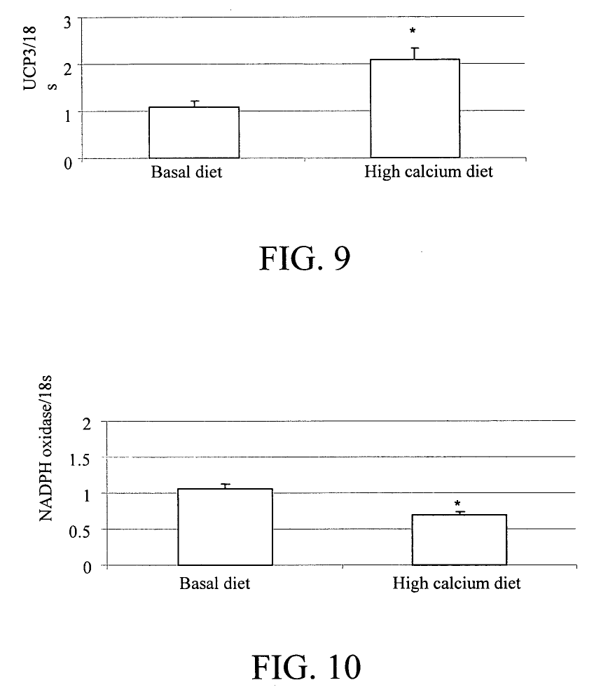

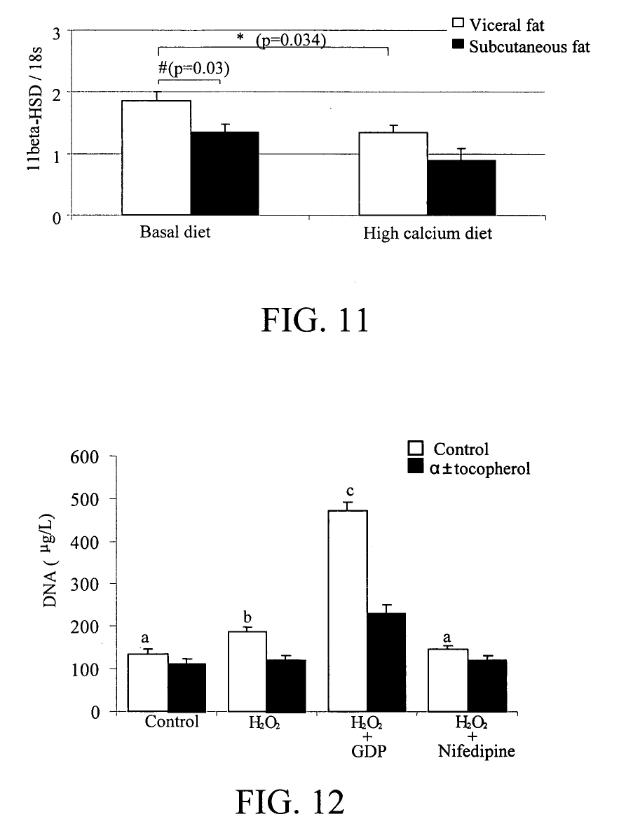

Based on the suitability of this model, we utilized aP2 transgenic mice as the animal to investigate the effect of dietary calcium in regulation of diet-induced oxidative stress in a three-week obesity induction period on high sucrose/high fat diets with either low calcium (0.4% from CaCO₃)(basal diet) or high calcium (1.2% from CaCO₃)(high calcium diet) content. Although feeding high fat/high sucrose diets ad libitum for 3 weeks induced weight and fat gain in all animals, mice on the high calcium diet gained only 50% of the body weight (p=0.04) and fat (p<0.001) as mice on the basal diet (FIG. 3). The high calcium diet also suppressed diet-induced hyperglycemic and reduced fasting blood glucose by 15% compared to mice on basal diet (p=0.003) (FIG. 4). The high calcium diet significantly reduced adipose intracellular ROS production by 64% and 18% (p<0.001) in visceral and subcutaneous adipose tissue respectively (FIG. 5). Consistent with this, the high calcium diet also inhibited adipose tissue NADPH oxidase expression, by 49% (p=0.012) in visceral adipose tissue and by 63% (p=0.05) in subcutaneous adipose tissue, respectively, compared to mice on the basal diet (FIG. 6), indicating that dietary calcium may inhibit oxidative stress by suppressing cytosolic enzymatic ROS production. Moreover, adipocyte intracellular calcium ([Ca²⁺]i) levels, which were previously demonstrated to favor adipocyte ROS production, were markedly suppressed in mice on the high calcium diet by 73%-80% (p<0.001) versus mice on the basal diet (FIG. 7), suggesting a role of [Ca²⁺]i in regulation of oxidative stress by dietary calcium. Consistent with our previous study, the high calcium diet also induced 367% and 191% increases in adipose UCP2 expression (p<0.001) in visceral and subcutaneous adipose tissue respectively, compared to mice on the basal diet (FIG. 8). Moreover, the pattern of UCP3 expression and indices of ROS production in skeletal muscle was consistent with these findings. UCP3 expression was 22% higher (p=0.006) (FIG. 9) and NADPH oxidase expression was 36% lower (p=0.001) (FIG. 10) in soleus muscle of mice on the high calcium diet compared to mice on the low calcium diet, suggesting that increases in UCP2 and UCP3 expression in adipose tissue and muscle, respectively, of animals on high calcium diets may contribute to reduced ROS levels.

We have recently shown that 1α, 25(OH)₂D₃ promotes cortisol production by stimulating 11β-HSD expression in cultured human adipocytes (Morris et al., 2005). However, the effect of modulation of 1α, 25(OH)₂D₃ via dietary calcium on this gene expression in vivo had not been investigated. Data from the present study demonstrates that the high calcium diet suppressed 11β-HSD expression in visceral adipose tissue by 39% (p=0.034) compared to mice on the basal diet (FIG. 11). Interestingly, 11β-HSD expression in visceral fat was markedly higher than subcutaneous fat in mice on basal low calcium group (p=0.034) whereas no difference was observed between the fat depots in mice on the high calcium diet.

Discussion

Previous data from our laboratory demonstrate that dietary calcium exerts an anti-obesity effect via a 1α, 25-(OH)₂-D₃-mediated mechanism (Zemel, 2005a). We have reported that 1α, 25-(OH)₂-D₃ plays a direct role in the modulation adipocyte Ca²⁺ signaling, resulting in an increased lipogenesis and decreased lipolysis (Xue et al., 1998; Xue et al., 2000). In addition, 1α, 25-(OH)₂-D₃ also plays a role in regulating human adipocyte UCP2 expression, suggesting that the suppression of 1α, 25-(OH)₂-D₃ and the resulting up-regulation of UCP2 may contribute to increased rates of energy utilization (Shi et al., 2001; Shi et al., 2002). Accordingly, the suppression of 1α, 25-(OH)₂-D₃ by increasing dietary calcium attenuates adipocyte triglyceride accumulation and caused a net reduction in fat mass in both mice and humans in the absence of caloric restriction (Zemel et al., 2000), a marked augmentation of body weight and fat loss during energy restriction in both mice and humans (Zemel et al., 2000; Zemel, 2004), and a reduction in the rate of weight and fat regain following energy restriction in mice (Sun et al., 2004a). Given that obesity and related disorders are associated with increased oxidative stress, dietary calcium may play a role in modulating diet-induced oxidative stress. Data from the present study demonstrate that dietary calcium decreased diet-induced ROS production. Our previous data demonstrate that 1α, 25(OH)₂D₃ stimulates Ca²⁺ signaling and suppresses UCP2 expression on human and murine adipocytes (Shi et al., 2002; Sun et al., 2004) and suppresses UCP3 expression in skeletal muscle (Sun et al., 2004); accordingly, dietary calcium suppression of ROS production is likely due to suppression of circulating 1α, 25(OH)₂D₃ levels and resultant reductions in Ca²⁺ signaling and increases in UCP2 and UCP3 expression. Furthermore, dietary calcium also appeared to regulate cytosol enzymatic ROS production by inhibiting NADPH oxidase expression, which also contributes to cellular ROS production.

The interaction between ROS and calcium have been intensively investigated (Toescu 2004; Ermak et al., 2002; Miwa et al., 2003; Brookes 2005). Calcium signaling is essential for production of ROS, and elevated intracellular calcium ([Ca²⁺]i) activates ROS-generating enzymes, such as NADPH-oxidase and myeloperoxidase, as well as the formation of free radicals by the mitochondrial respiratory chain (Gordeeva et al., 2003). Interestingly, increased ROS production also stimulates [Ca²⁺]i by activating calcium channels on both the plasma membrane and endoplasmic reticulum (ER) (Volk et al., 1997). Thus, there is a bi-directional interaction wherein ROS cellular calcium homeostasis and calcium-dependent physiological processes while manipulation of calcium signaling may also regulate cellular ROS production. Consistent with this concept, the present data show that suppression [Ca²⁺]i by high dietary calcium was associated with amelioration of ROS production in adipose tissue.

Respiration is associated with production of ROS, and mitochondria produce a large fraction of the total ROS made in cells (Brand et al., 2004). Mild uncoupling of respiration diminishes mitochondrial ROS formation by dissipating mitochondrial proton gradient and potential (Miwa et al., 2003). Korshunov et al. has demonstrated that slight increase of the H+ backflux (to the matrix), which diminishes Δψ, results in a substantial decrease in mitochondrial ROS formation (Korshunov et al., 1997). Accordingly, the H+ backflow induced by uncoupling via UCPs would be expected to down-regulate ROS production. Mild activation of UCPs may therefore play a role in the antioxidant defense system and it is reasonable to propose that dietary calcium induced suppression of 1α, 25-(OH)₂D₃, which has been demonstrated to inhibit UCP2 expression (Shi et al., 2002), may inhibit ROS production. Indeed, in the present study, we have shown that high dietary calcium up-regulated both UCP2 expression in adipose tissue and UCP3 expression in skeletal muscle, and these findings were associated decreased ROS production, indicating a role of mitochondrial uncoupling in regulation of oxidative stress.

We also compared the ROS production between subcutaneous and visceral adipose tissue. Consistent with our previous data (Zemel, 2005a; Zemel et al., 2005a), animals on the basal low calcium diet showed markedly higher visceral fat gain than subcutaneous fat versus mice on the high calcium diet (data not shown) and exhibited strikingly enhanced ROS production and NADPH oxidase expression in visceral fat versus subcutaneous fat. Conversely, high dietary calcium ameliorated visceral fat gain and mice on the high calcium diet showed no significantly greater ROS production in visceral fat versus subcutaneous fat. These results therefore indicated that higher visceral fat predisposes to enhanced ROS production. Accordingly, we further evaluated the involvement of glucocorticoid by measuring 11β hydroxysteroid dehydrogenase (11β-HSD) expression, the key enzyme responsible for converting glucocorticoid into its active form (Agarwal 2003). We demonstrated that 11β-HSD expression in visceral fat was markedly higher than subcutaneous fat in mice on basal low calcium group whereas no difference was observed between the fat depots in mice on the high calcium diet. We also found the high calcium diet suppressed 11β-HSD expression in visceral adipose tissue compared to mice on the low calcium diet. These findings demonstrated that dietary calcium exerts greater effect on inhibition of visceral fat gain via suppressing formation of active glucocoticoid and thus explained the markedly decreased visceral fat gain in mice on the high calcium diet than mice on the low calcium diet. Therefore, the enhanced ROS production observed in visceral fat compare to subcutaneous fat in response to the high fat/high sucrose diet only in mice on low calcium diet suggested that suppression of ROS production by dietary calcium may be mediated, at least in part, by the regulation of glucocorticoid associated fat distribution. We recently reported in vitro observation that 1α, 25 (OH)₂D₃ directly regulates adipocyte 11β-HSD 1 expression and local cortisol levels in cultured human adipocytes (Morris et al., 2005), and data from this study provides the first in vivo evidence that dietary calcium may contribute to the preferential loss of visceral adiposity and obesity associated oxidative stress by regulating adipose tissue 11β-HSD expression and glucocorticoid production.

In conclusion, these data support a role for dietary calcium in the regulation of diet- and obesity-induced oxidative stress. Potential mechanisms include increases in UCP2 and UCP3 expression, suppression of [Ca²⁺]i, and/or inhibition of NADPH oxidase and 11β-HSD gene expression. These data also support our previous observation that dietary calcium inhibits obesity, with partially selective effects on visceral adipose tissue, and leads to significant changes in adipose tissue metabolism, including accelerated adipose tissue deposition and reduced ROS production.

### EXAMPLE 2

### 1, 25-Dihydroxyvitamin D Modulation of Reactive Oxygen Species Production and Cell Proliferation in Human and Murine Adipocytes

3T3-L1 preadipocytes were incubated at a density of 8000 cells/cm² (10 cm² dish) and grown in Dulbecco's modified Eagle's medium (DMEM) containing 10% FBS and antibiotics (adipocyte medium) at 37° C. in 5% CO₂ in air. Confluent preadipocytes were induced to differentiate with a standard differentiation medium consisting of DMEM-F 10 (1:1, vol/vol) medium supplemented with 1% FBS, 1 μM dexamethasone, IBMX (0.5 mM) and antibiotics (1% Penicillin-Streptomycin). Preadipocytes were maintained in this differentiation medium for 3 days and subsequently cultured in adipocyte medium. Cultures were re-fed every 2-3 days to allow 90% of cells to reach full differentiation before conducting chemical treatment. Chemicals were freshly diluted in adipocyte medium before treatment. Cells were washed with fresh adipocyte medium, re-fed with medium containing the different treatments, and incubated at 37° C. in 5% CO₂ in air before analysis. Cell viability was measured via trypan blue exclusion.

Human preadipocytes used in this study were supplied by Zen-Bio (Research Triangle, N.C.). Preadipocytes were inoculated in DMEM/Ham's F-10 medium (DMEM-F10) (1:1, vol/vol) containing 10% FBS, 15 mmol/L HEPES, and antibiotics at a density of 30,000 cells/cm². Confluent monolayers of preadipocytes were induced to differentiate with a standard differentiation medium consisting of DMEM-F10 (1:1, vol/vol) medium supplemented with 15 mmol/L HEPES, 3% FBS, 33 μmol/L biotin, 17 μmol/L pantothenate, 100 nmol/L insulin, 0.25 μmol/L methylisobutylxanthine (MIX), 1 μmol/L dexamethasone, 1 μmol/L BRL49653, and antibiotics. Preadipocytes were maintained in this differentiation medium for 3 days and subsequently cultured in adipocyte medium in which BRL49653 and MIX were omitted. Cultures were refed every 2-3 days.

UCP2 Transfection

UCP2 full-length cDNAs was amplified by RT-PCR using mRNAs isolated from mouse white adipose tissues. The PCR primers for this amplification are shown as follows: UCP2 forward, 5′-GCTAGCATGGTTGGTTTCAAG-3′ (SEQ ID NO: 1), reverse, 5′-GCTAGCTCAGAAAGGTGAATC-3′ (SEQ ID NO: 2). The PCR products were then subcloned into pcDNA4/His expression vectors. The linearized constructs were transfected into 3T3-L1 preadipocytes using lipofectamine plus standard protocol (Invitrogen, Carlsbad, Calif.). After 48 hrs of transfection, cells were split and cultured in selective medium containing 400 μg/ml zeocin for the selection of resistant colonies. Cells were fed with selective medium every 3 days until resistant colonies could be identified. These resistant foci were picked, expanded, tested for expression, and frozen for future experiments.

Determination of Mitochondrial Membrane Potential

Mitochondrial membrane potential was analyzed fluorometrically with a lipophilic cationic dye JC-1 (5,5′,6,6′-tetrachloro-1,1′,3,3′-tetraethylbenzimidazol carbocyanine iodide) using a mitochondrial potential detection kit (Biocarta, San Diego, Calif.). Mitochondrial potential was determined as the ratio of red fluorescence (excitation 550 nm, emission 600 mn) and green fluorescence (excitation 485 nm, emission 535 nm) using a fluorescence microplate reader.

Measurement of Intracellular Ca²⁺([Ca²⁺]i)

[Ca²⁺]i in adipocytes was measured using a fura-2 dual-wavelength fluorescence imaging system. Cells were plated in 35-mm dishes (P35G-0-14-C, MatTek). Prior to [Ca²⁺]i measurement, cells were put in serum-free medium overnight and rinsed with HEPES balanced salt solution (HBSS) containing the following components (in mmol/L): 138 NaCl, 1.8 CaCl₂, 0.8 MgSO₄, 0.9 NaH₂PO₄, 4 NaHCO₃, 5 glucose, 6 glutamine, 20 HEPES, and 1% bovine serum albumin. Cells were loaded with fura-2 acetoxymethyl ester (fura-2 AM) (10 μmol/L) in the same buffer for 2 h at 37° C. in a dark incubator with 5% CO₂. To remove extracellular dye, cells were rinsed with HBSS three times and then post-incubated at room temperature for an additional 1 h for complete hydrolysis of cytoplasmic fura-2 AM. The dishes with dye-loaded cells were mounted on the stage of Nikon TMS-F fluorescence inverted microscope with a Cohu model 4915 charge-coupled device (CCD) camera. Fluorescent images were captured alternatively at excitation wavelengths of 340 and 380 nm with an emission wavelength of 520 nm. After establishment of a stable baseline, the responses to 1α, 25-(OH)₂-D₃ was determined. [Ca²⁺]i was calculated using a ratio equation as described previously. Each analysis evaluated responses of 5 representative whole cells. Images were analyzed with InCyt Im2 version 4.62 imaging software (Intracellular Imaging, Cincinnati, Ohio). Images were calibrated using a fura-2 calcium imaging calibration kit (Molecular Probes, Eugene, Oreg.) to create a calibration curve in solution, and cellular calibration was accomplished using digitonin (25 μmol/L) and pH 8.7 Tris-EGTA (100 mmol/L) to measure maximal and minimal [Ca²⁺]i levels respectively.

Total RNA Extraction

A total cellular RNA isolation kit (Ambion, Austin, Tex.) was used to extract total RNA from cells according to manufacturer's instruction.

Quantitative Real Time PCR

Adipocyte 18s, cyclin A, NADPH oxidase, and UCP2 were quantitatively measured using a Smart Cycler Real Time PCR System (Cepheid, Sunnyvale, Calif.) with a TaqMan 1000 Core Reagent Kit (Applied Biosystems, Branchburg, N.J.). The primers and probe sets were ordered from Applied Biosystems TaqMan® Assays-on-Demand™ Gene Expression primers and probe set collection according to manufacture's instruction. Pooled adipocyte total RNA was serial-diluted in the range of 1.5625-25 ng and used to establish a standard curve; total RNAs for unknown samples were also diluted in this range. Reactions of quantitative RT-PCR for standards and unknown samples were also performed according to the instructions of Smart Cycler System (Cepheid, Sunnyvale, Calif.) and TaqMan Real Time PCR Core Kit (Applied Biosystems, Branchburg, N.J.). The mRNA quantitation for each sample was further normalized using the corresponding 18s quantitation.

Assessment of cell Proliferation

Cells were plated in DMEM with different treatment in duplicate in 96-well plates. After 48 h, a CyQUANT Cell Proliferation Kit (Molecular Probes, Eugene, Oreg.) was used following the manufacturer's protocol a microplate fluorometer (Packard Instrument Company, Inc., Downers Grove, Ill.) was used to measure CyQUANT fluorescence. Cell viability was determined by Trypan blue exclusion examination.

Determination of Intracellular ROS Generation

Intracellular ROS generation was assessed using 6-carboxy-2′,7′-dichlorodihydrofluorescein diacetate (H2-DCFDA) as described previously (Manea et al., 2004). Cells were loaded with H2-DCFDA (2 μmol/L) 30 minute before the end of the incubation period (48 h). After washing twice with PBS, cells were scraped and disrupted by sonication on ice (20s). Fluorescence (emission 543 nm or 527 nm) and DNA content were then measured as described previously. The intensity of fluorescence was expressed as arbitary units per ng DNA.

Statistical Analysis

All data are expressed as mean±SEM. Data were evaluated for statistical significance by analysis of variance (ANOVA), and significantly different group means were then separated by the least significant difference test by using SPSS (SPSS Inc, Chicago, Ill.).

Results

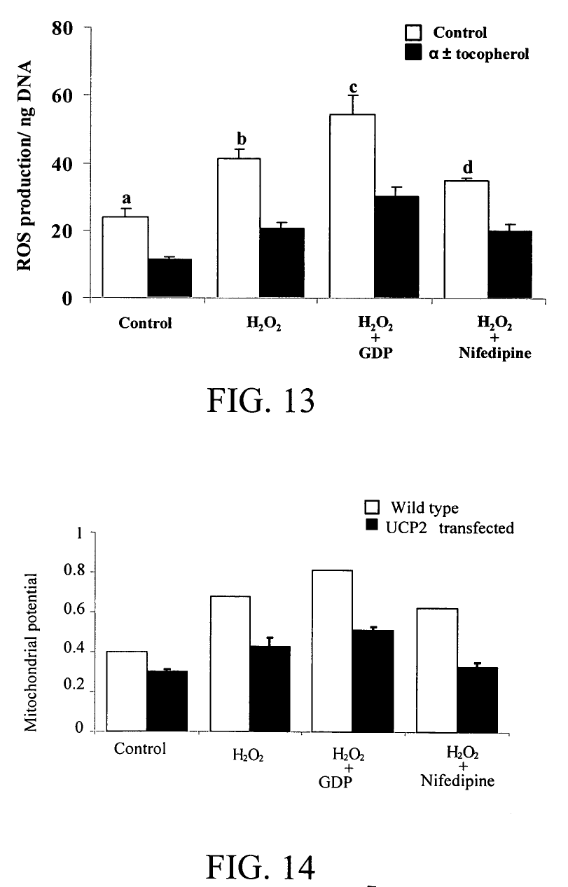

Our first aim was to examine whether ROS have effect on adipocyte proliferation. The data presented in FIG. 12 indicate that this is indeed the case. Treatment of 3T3-L1 adipocytes with H₂O₂ increased the total DNA of cultured cells by 39% (p<0.001), while addition of antioxidant α-tocopherol completely blocked this effect. The effect of ROS on adipocyte proliferation appears to be regulated by mitochondrial uncoupling and intracellular calcium homeostasis. Addition of mitochondrial uncoupling inhibitor GDP augmented the stimulation of cell proliferation by H₂O₂ by 183% (p<0.005) while calcium channel antagonist nifedipine had the opposite effect and suppressed H₂O₂ induced cell DNA synthesis (p<0.05). Since inhibiting mitochondrial uncoupling and increasing [Ca²⁺]i have been demonstrated to contribute to increased ROS production, GDP may increases DNA synthesis by increasing ROS production while nifedipine exerts the opposite effect via suppression of ROS production. Consistent with this, FIG. 13 shows that addition of GDP increased ROS production by 24% (p<0.01) compare H₂O₂ treatment alone while nifedipine inhibited H₂O₂ induced ROS production by 25% (p<0.003). FIGS. 12 and 13 also demonstrate that addition of antioxidant α-tocopherol inhibited both ROS production and DNA synthesis in all groups. These results suggest that ROS stimulated cell proliferation in cultured adipocytes and that this effect can be regulated by mitochondrial uncoupling status and intracellular homeostasis. Similar results were also observed in human adipocytes (data not shown).

To further investigate the interaction between ROS and mitochondrial uncoupling status, we measured mitochondrial potential in both wild-type 3T3-L1 adipocytes and UCP2 transfected 3T3-L1 adipocytes. FIG. 14 demonstrates that H₂O₂ increased mitochondrial potential by 72% and that addition of GDP augmented this effect by 10%, indicating that ROS production inhibits mitochondrial uncoupling. Nifedipine suppressed the H₂O₂ induced increase in mitochondrial potential and this result confirms that calcium channel antagonist inhibits ROS production. UCP2 transfection increased mitochondrial potential and suppressed the effect of H₂O₂ on mitochondrial uncoupling, indicating that ROS production is regulated, in part by mitochondrial potential and UCP2.

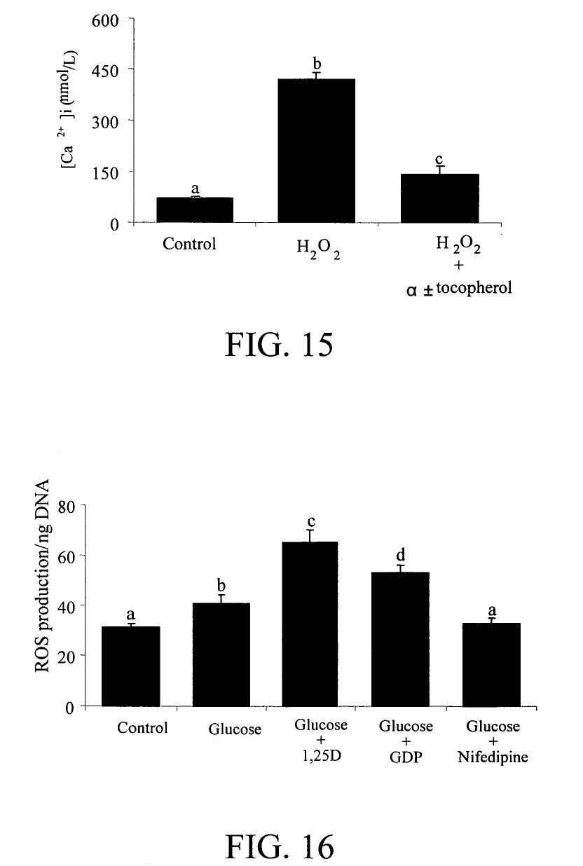

FIG. 15 demonstrates that ROS has a direct role in regulation of intracellular calcium homeostasis in 3T3-L1 adipocytes. H₂O₂ induced a 5-fold increase in [Ca²⁺]i (p<0.001) and this effect was reversed by addition of antioxidant α-tocopherol. Since suppression of intracellular calcium influx by nifedipine decreased ROS production as described in FIG. 13, this result suggests a positive feedback interaction between ROS production and intracellular calcium homeostasis: ROS stimulate [Ca²⁺]i and elevated [Ca²⁺]i also favors ROS production. Similar results were observed in Zen-Bio human adipocytes (data not shown).

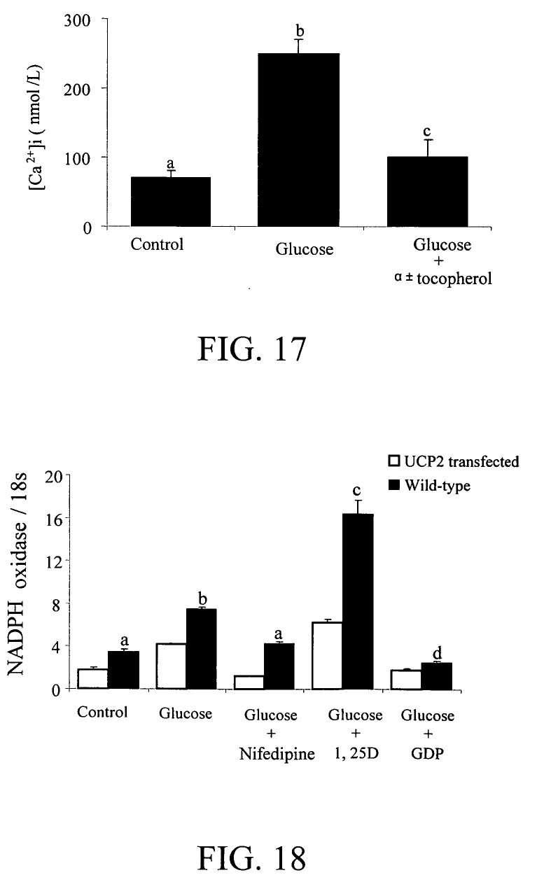

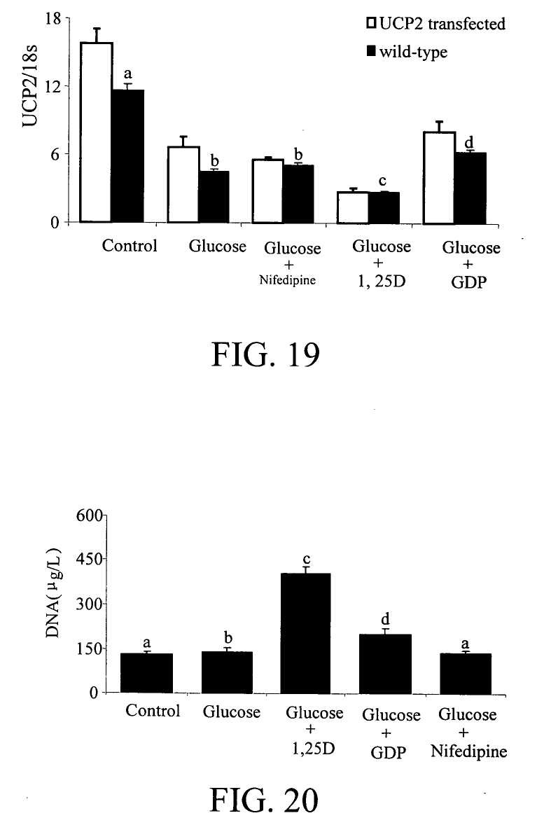

Hyperglycemia is one of the most common clinical signs in obesity and diabetes, which has been demonstrated to be associated with increased ROS production. Accordingly, we next investigated the effect and mechanism of high glucose level on ROS production and consequent adipocyte proliferation. As shown in FIG. 16, high glucose treatment increased ROS production significantly (p<0.05) and this effect was partially reversed by addition of nifedipine. Addition of GDP further stimulated ROS production compared to glucose alone. Notably, treatment of adipocytes with 1α, 25-(OH)₂D₃, which was previously found to suppress mitochondrial uncoupling and to increase [Ca²⁺]i in adipocytes, resulted in greater stimulation of ROS production than either glucose alone or glucose plus GDP (p<0.05), suggesting that 1α, 25-(OH)₂D₃ stimulates ROS production by both inhibition of mitochondrial uncoupling and stimulation of [Ca²⁺]i. Glucose also increased [Ca²⁺]i by 3-fold (p<0.001) (FIG. 17) and this effect was partially blocked by addition of α-tocopherol, indicating that stimulation of [Ca²⁺]i by high glucose is partially attributable to ROS production. Consistent with this, FIG. 18 shows that high glucose also increased expression of NADPH oxidase (p<0.001), a key enzyme in ROS production, in both wild-type and UCP2 transfected 3T3-L1 adipocytes, but UCP2 overexpression attenuated this effect. These results suggest that high glucose may increase ROS production by stimulating NADPH oxidase expression. Addition of 1α, 25-(OH)₂D₃ stimulated NADPH oxidase expression while nifedipine suppressed its expression. Although GDP has been shown to increases ROS production, we found GDP suppressed NADPH oxidase expression, indicating that regulation of ROS production by GDP is not via up-regulation of ROS-generating enzyme gene expression. FIG. 19 provides further evidence for the role of UCP2 in the regulation high glucose induced ROS production. High glucose inhibits UCP2 expression in both wild type and UCP2 transfected adipocytes, indicating that high glucose stimulates ROS production by regulating mitochondrial uncoupling status.

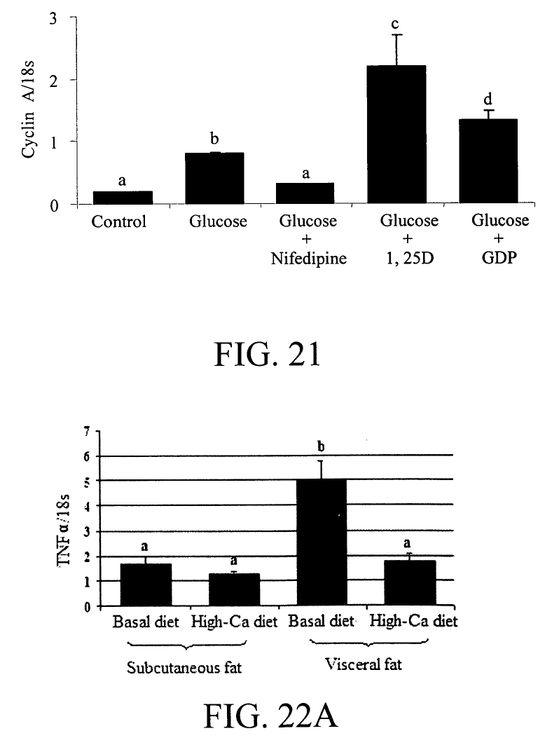

FIG. 20 demonstrates that stimulation of ROS production by high glucose is associated with increased DNA synthesis. High glucose alone significantly increased DNA synthesis (p<0.03) and this effect was by augmented by addition of GDP or 1α, 25-(OH)₂D₃. In contrast, inhibition of ROS production by nifedipine decreased glucose induced DNA synthesis (p<0.05). To further investigate the effect of high glucose on adipocyte proliferation, we also observed the expression of cyclin A (FIG. 21). Consistent with the DNA synthesis data, high glucose stimulated cyclin A expression by 3-fold (p<0.001), and GDP and 1α, 25-(OH)₂D₃ augmented this effect while nifedipine suppressed its expression. These data suggest high glucose stimulates adipocyte proliferation and this effect may be at least partially mediated by its stimulation of ROS production.

Discussion

Obesity and diabetes are associated with increased oxidative stress, and ROS may play a role in regulation of adipocyte proliferation. In the present study, we demonstrated that a low concentration of H₂O₂ stimulates cell proliferation in cultured adipocytes. This effect can be augmented by a mitochondrial uncoupling inhibitor and suppressed by a calcium channel antagonist, indicating that mitochondrial potential and intracellular calcium homeostasis may play a role in regulation of ROS induced cell proliferation. 1α, 25-(OH)₂D₃, which has been demonstrated to stimulate [Ca²⁺]i and to inhibit UCP2 expression, stimulates ROS production and cell proliferation in adipocytes. High glucose also exerts stimulatory effect on ROS production and this effect can be augmented by addition of 1α, 25-(OH)₂D₃, suggesting that 1α, 25-(OH)₂D₃ may involved in regulation of ROS production in adipocytes. These results indicate that strategies to suppress 1α, 25-(OH)₂D₃ levels, such as increasing dietary calcium, may reduce oxidative stress and thereby inhibit ROS-induced stimulation of adipocyte proliferation.

Elevated oxidative stress has been reported in both humans and animal models of obesity (Sonta et al., 2004; Atabek et al., 2004), suggesting that ROS may play a critical role in the mechanisms underlying proliferative responses. This concept is supported by evidence that both H₂O₂ and superoxide anion induce mitogenesis and cell proliferation in several mammalian cell types (Burdon 1995). Furthermore, reduction of oxidants via supplementation with antioxidants inhibits cell proliferation in vitro (Khan et al., 2004; Simeone et al., 2004). Although the mechanisms for the involvement of oxidative stress in the induction of cell proliferation are not known, it has been demonstrated that ROS and other free radicals influence the expression of number of genes and transduction pathways involved in cell growth and proliferation. The most significant effects of oxidant on signaling pathways have been observed in the mitogen-activated protein (MAP) kinase/AP1, and it has been suggested that ROS can activate MAP kinases and thereby transcription factors activator protein-1(AP-1) (Chang et al., 2001), a collection of dimeric basic region-leucine zipper proteins which activates cyclin-dependent kinase and entry into cell division cycle (Kouzarides et al., 1989). Furthermore, the elevation of cytosolic calcium level induced by ROS results in activation of protein kinase C (PKC) required for expression of positive regulators of cell proliferation such as c-fos and c-jun (Lin 2004; Amstad et al., 1992; Hollander et al., 1989). ROS have also been implicated as a second messenger involved in activation of NF-κB (Song et al., 2004), whose expression has been shown to stimulate cell proliferation via tumor necrosis factor (TNF) and interleukin-1 (IL-1) (Giri et al., 1998). The effect of ROS on NF-κB activation is further supported by studies which demonstrated that expression NF-κB can be suppressed by antioxidants (Nomura et al., 2000; Schulze-Osthoff et al., 1997). In addition, ROS can modify DNA methylation and cause oxidative DNA damage, which result in decreased methylation patterns (Weitzman et al., 1994) and consequently contribute to an overall aberrant gene expression. ROS may also attribute to the inhibition of cell-to-cell communication and this effect can result in decreased regulation of homeostatic growth control of normal surrounding cells and lead to clonal expansion (Cerutti et al., 1994; Upham et al., 1997). Despite these mechanisms proposed to explain the stimulatory effect on cell proliferation, limited studies have been conducted on adipocytes. In present study, we demonstrated that low concentrations of ROS promote cell proliferation in cultured human and murine adipocytes. However, further investigation for the underlying molecular mechanisms is required.

The yield of ROS can be efficiently modulated by mitochondrial uncoupling. Korshunov et al. has demonstrated that slight increase of the H+ backflux (to the matrix), which diminishes Δψ, results in a substantial decrease of mitochondrial ROS formation (Korshunov et al., 1997). Accordingly, the H⁺ backflow from UCP-induced uncoupling would be expected to down-regulate ROS production. In addition, calcium can active ROS-generating enzymes directly and activation of calcium dependent PKC favors assembly of the active NADPH-oxidase complex (Gordeeva et al., 2003), indicating that [Ca²⁺]i may be another key player in regulation of ROS production. Accordingly, it is reasonable to propose that 1α, 25-(OH)₂D₃, which has been demonstrated both to inhibit mitochondrial uncoupling and to stimulate [Ca²⁺]i in adipocytes, would stimulate ROS production and may consequently be involved in the regulation of adipocyte proliferation. Indeed, in the present study, we have shown that addition of 1α, 25-(OH)₂D₃ augmented high glucose-induced ROS production and adipocyte proliferation. This effect was further enhanced by a mitochondrial uncoupling inhibitor and suppressed by calcium channel antagonism, indicating that 1α, 25-(OH)₂D₃ stimulates ROS production by increasing [Ca²⁺]i and by inhibiting mitochondrial uncoupling. Furthermore, previous studies suggest that 1α, 25-(OH)₂D₃ may act as an prooxidant in various cell types (Koren et al., 2001) and treatment with 1α, 25-(OH)₂D₃ inhibited the expression of the major constituents of the cellular defense system against ROS (Banakar et al., 2004).

Previous data from our laboratory have demonstrated that 1α, 25-(OH)₂-D₃ appears to modulate adipocyte lipid and energy metabolism via both genomic and non-genomic pathways (Zemel, 2004; Shi et al., 2001; Shi et al., 2002). We have reported that 1α, 25-(OH)₂-D₃ plays a direct role in the modulation adipocyte Ca²⁺ signaling, resulting in an increased lipogenesis and decreased lipolysis (Shi et al., 2001). In addition, 1α, 25-(OH)₂-D₃ also plays a role in regulating human adipocyte UCP2 mRNA and protein levels, indicating that the suppression of 1α, 25-(OH)₂-D₃ and the resulting up-regulation of UCP2 may contribute to increased rates of lipid oxidation (Shi et al., 2002). In addition, we also demonstrate that physiological doses of 1α, 25-(OH)₂-D₃ inhibit apoptosis in differentiated human and 3T3-L1 adipocytes (Sun et al., 2004b), and that the suppression of 1α, 25-(OH)2-D₃ in vivo by increasing dietary calcium stimulates adipocyte apoptosis in aP2 transgenic mice (Sun et al., 2004b), suggesting that the stimulation of adipocyte apoptosis contributes to the observed reduction in adipose tissue mass after administration of high calcium diets (Shi et al., 2002). Accordingly, the suppression of 1α, 25-(OH)₂-D₃ by increasing dietary calcium attenuates adipocyte triglyceride accumulation and caused a net reduction in fat mass in both mice and humans in the absence of caloric restriction (Zemel et al., 2000), a marked augmentation of body weight and fat loss during energy restriction in both mice and humans (Zemel et al., 2000; Zemel et al., 2004), and a reduction in the rate of weight and fat regain following energy restriction in mice (Sun et al., 2004). Data from present study provide further evidence to support the role of 1α, 25-(OH)₂D₃ in favoring energy storage and fat mass expansion by stimulating ROS production and adipocyte proliferation. ROS stimulates adipocyte proliferation and this effect can by suppressed by mitochondrial uncoupling and stimulated by elevation of intracellular calcium. 1α, 25-(OH)₂D₃ increases ROS production by inhibiting UCP2 expression and increasing [Ca²⁺]i and consequently favors adipocyte proliferation. Accordingly, the present data suggest that suppression 1α, 25-(OH)₂D₃ by increasing dietary calcium may reduce 1α, 25-(OH)₂D₃ mediated ROS production and limit ROS induced adipocyte proliferation, resulting in reduced adiposity.

This work demonstrated a direct effect of oxidative stress on adipocyte proliferation in white adipose tissue and this observation may have important implications in understanding the adipose mass changes observed under oxidative stress. However, cell proliferation was only evaluated by DNA content and cyclin expression level. Further, various sources of ROS production may play different roles in regulation of cell signaling in cell cycle and cell metabolism. Although we demonstrated both mitochondrial ROS production and cellular enzymatic ROS production are associated with adipocyte proliferation, the contribution of each source needs further investigation.

### EXAMPLE 3

### Calcium and 1, 25-(OH)₂-D₃ Regulation of Adipokine Expression in Murine and Human Adipocytes and aP2-agouti Transgenic Mice

### Materials and Methods

Animals and Diets

At 6 wk of age, 20 male aP2-agouti transgenic mice from our colony were randomly divided into two groups (10 mice/group) and fed a modified AIN 93 G diet with suboptimal calcium (0.4% from calcium carbonate) or high calcium (1.2% from calcium carbonate) respectively. Sucrose was the sole carbohydrate source, providing 64% of energy, and fat was increased to 25% of energy with lard. Mice were studied for three weeks, during which food intake and spillage were measured daily and body weight, fasting blood glucose, food consumption assessed weekly. At the conclusion of the study, all mice were killed under isofluorane anesthesia and blood collected via cardiac puncture; visceral fat pads (perirenal and abdominal), subcutaneous fat pads (subscapular) and soleus muscle were immediately excised, weighed and used for further study, as described below.

This study was approved from an ethical standpoint by the Institutional Care and Use Committee of The University of Tennessee.

### Cell Culture

3T3-L1 pre-adipocytes were incubated at a density of 8000 cells/cm² (10 cm² dish) and grown in Dulbecco's modified Eagle's medium (DMEM) containing 10% FBS and antibiotics (adipocyte medium) at 37° C. in 5% CO₂ in air. Confluent pre-adipocytes were induced to differentiate with a standard differentiation medium consisting of DMEM-F10 (1:1, vol/vol) medium supplemented with 1% fetal bovine serum (FBS), 1 μM dexamethasone, isobutylmethylxanthine (IBMX) (0.5 mM) and antibiotics (1% Penicillin-Streptomycin). Pre-adipocytes were maintained in this differentiation medium for 3 days and subsequently cultured in adipocyte medium. Cultures were re-fed every 2-3 days to allow 90% cells to reach fully differentiation before conducting chemical treatment.

Human pre-adipocytes used in this study were supplied by Zen-Bio (Research Triangle, N.C.). Preadipocytes were inoculated in DMEM/Ham's F-10 medium (DMEM-F10) (1:1, vol/vol) containing 10% FBS, 15 mmol/L 4-2-hydroxyethyl-1-piperazineethanesulfonic acid (HEPES), and antibiotics at a density of 30,000 cells/cm². The cells are isolated from the stromal vascular fraction of human subcutaneous adipose tissue and differentiated in vitro as follows: Confluent monolayers of pre-adipocytes were induced to differentiate with a standard differentiation medium consisting of DMEM-F10 (1:1, vol/vol) medium supplemented with 15 mmol/L HEPES, 3% FBS, 33 μmol/L biotin, 17 μmol/L pantothenate, 100 nmol/L insulin, 0.25 μmol/L methylisobutylxanthine, 1 μmol/L dexamethasone, 1 μmol/L BRL49653, and antibiotics. Preadipocytes were maintained in this differentiation medium for 3 days and subsequently cultured in adipocyte medium in which BRL49653 and MIX were omitted. Cultures were re-fed every 2-3 days till fully differentiated.

Cells were incubated in serum free medium overnight before chemical treatment. Chemicals were freshly diluted in adipocyte medium before treatment. Cells were washed with fresh adipocyte medium, re-fed with medium containing the different treatments (control, 10 nmol/L 1α, 25-(OH)₂-D₃, 10 μmol/L nifedipine, 10 nmol/L 1α, 25-(OH)₂-D₃ plus 10 μmol/L nifepipine, 100 nmol/L H₂O₂, 1 μmol/L α±tocopherol, or 100 nmol/L H₂O₂ plus 1 μmol/L α±tocopherol) and incubated at 37° C. in 5% CO₂ for 48 h in air before analysis. Cell viability was measured via trypan blue exclusion.

Total RNA Extraction

A total cellular RNA isolation kit (Ambion, Austin, Tex.) was used to extract total RNA from cells according to manufacturer's instruction.

Plasma 1α, 25-(OH)₂-D₃ Assay

A 1α, 25-(OH)₂-D₃-vitamin D ELISA kit was used to measure plasma 1α, 25-(OH)₂-D₃ content according to the manufacturer's instructions (Alpco Diagnostics, Windham, N.H.).

Quantitative Real Time PCR

Adipocyte and muscle 18s, TNFα, IL-6, IL-8, IL-15 and adiponectin were quantitatively measured using a smart cycler real-time PCR system (Cepheid, Sunnyvale, Calif.) with a TaqMan 1000 Core Reagent Kit (Applied Biosystems, Branchburg, N.J.). The primers and probe sets were obtained from Applied Biosystems TaqMan® Assays-on-Demand™ Gene Expression primers and probe set collection and utilized according to manufacture's instructions. Pooled adipocyte total RNA was serial-diluted in the range of 1.5625-25 ng and used to establish a standard curve; and total RNA for the unknown samples were also diluted in this range. Reactions of quantitative RT-PCR for standards and unknown samples were also performed according to the instructions of Smart Cycler System (Cepheid, Sunnyvale, Calif.) and TaqMan Real Time PCR Core Kit (Applied Biosystems, Branchburg, N.J.). The mRNA quantitation for each sample was further normalized using the corresponding 18s quantitation.

Statistical Analysis

Data were evaluated for statistical significance by analysis of variance (ANOVA) or t-test, and significantly different group means were then separated by the least significant difference test by using SPSS (SPSS Inc, Chicago, Ill.). All data presented are expressed as mean±SEM.

Results

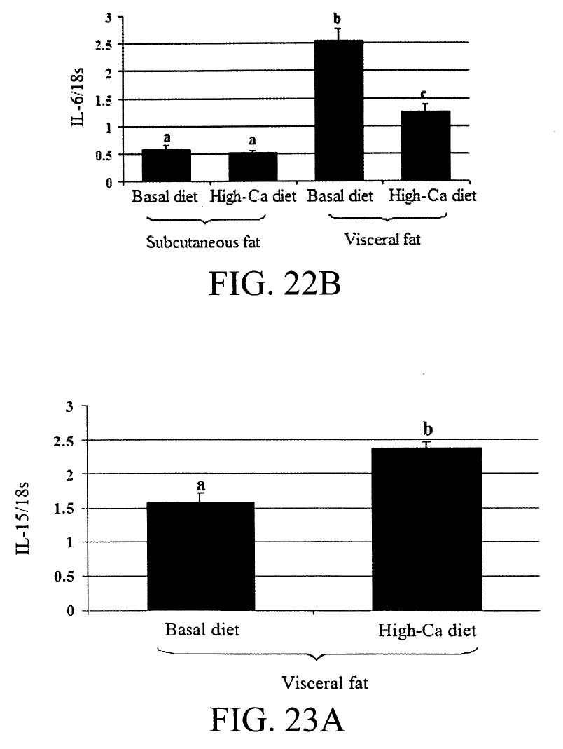

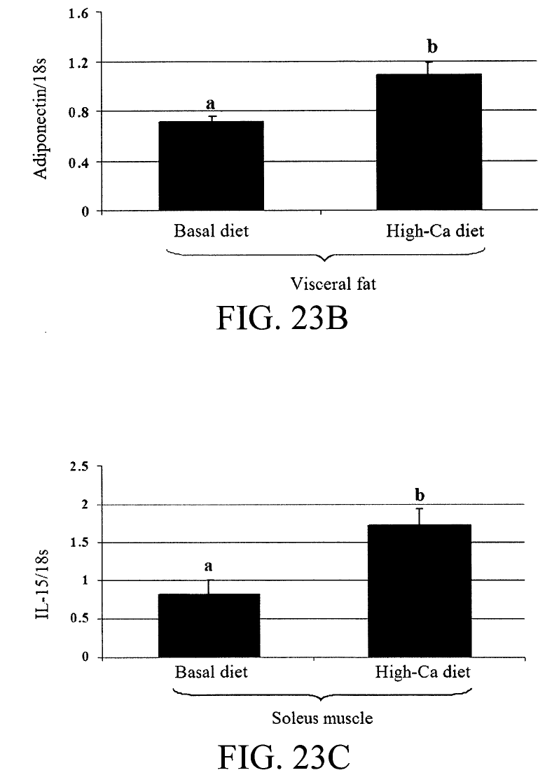

Dietary calcium regulates inflammatory cytokine production in adipose tissue and skeletal muscle. Feeding the high calcium ad libitum for 3 weeks significantly decreased weight and fat gain (Table 1) and suppressed TNFα gene expression by 64% in visceral, but not subcutaneous, fat compared with mice on low calcium basal diet (FIG. 22A)(p<0.001). Similarly, IL-6 expression was decreased by 51% in visceral fat of mice on the high calcium diet versus mice on the low calcium basal diet (FIG. 22B) (p<0.001) and this effect was absent in subcutaneous fat. In contrast, dietary calcium up-regulated IL-15 expression in visceral fat, with a 52% increases in mice on high calcium diet compared with animals on low calcium diet (FIG. 23A) (p=0.001). Adiponectin expression was similarly elevated in visceral fat of mice on the high calcium diet versus mice on low calcium diet (FIG. 23B) (p=0.025). The high calcium diet also induced a 2-fold increase in IL-15 expression in soleus muscle compared with mice on low calcium diet (FIG. 23C) (p=0.01).

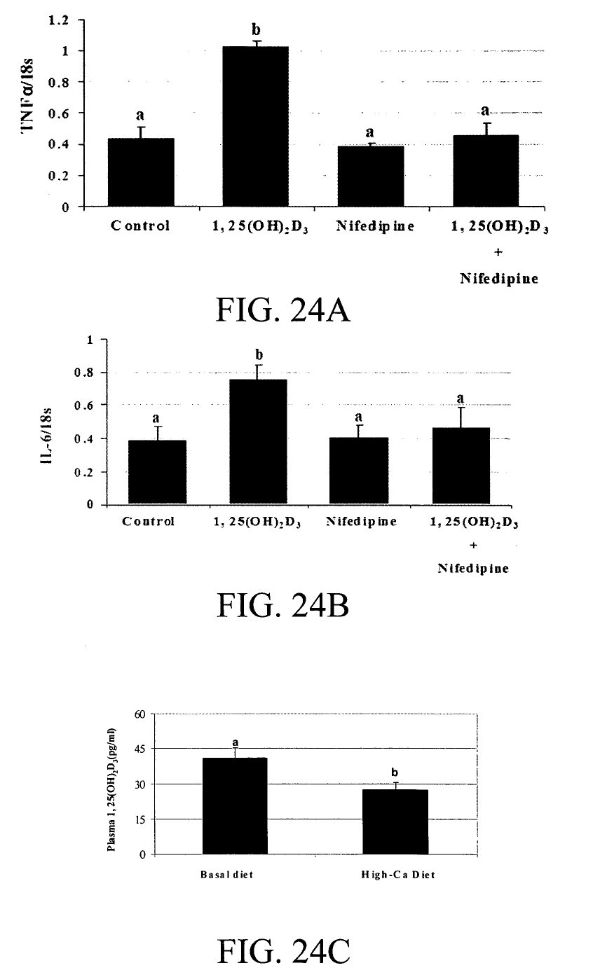

Intracellular calcium and 1α, 25-(OH)₂-D₃ regulates cytokine production in cultured murine and human adipocytes. We investigated the role of 1α, 25-(OH)₂-D₃ and calcium in regulation of adipokine production in vitro. FIG. 24A shows that 1α, 25-(OH)₂-D₃ stimulated TNFα expression by 135% in 3T3-L1 adipocyte and addition of calcium channel antagonist nifedipine completely blocked this effect (p<0.001), while nifedipine alone exerted no effect. Similarly, 1α, 25-(OH)₂-D₃ markedly increased IL-6 expression in 3T3-L1 adipocyte and this effect was reversed by addition of nifedipine (p=0.016) (FIG. 24B). Similar results were observed in human adipocytes (data not shown). These data suggested that 1α, 25-(OH)₂-D3 stimulated cytokine production by increasing intracellular calcium influx. The high calcium diet suppressed plasma 1α, 25-(OH)₂-D₃ (FIG. 24C).

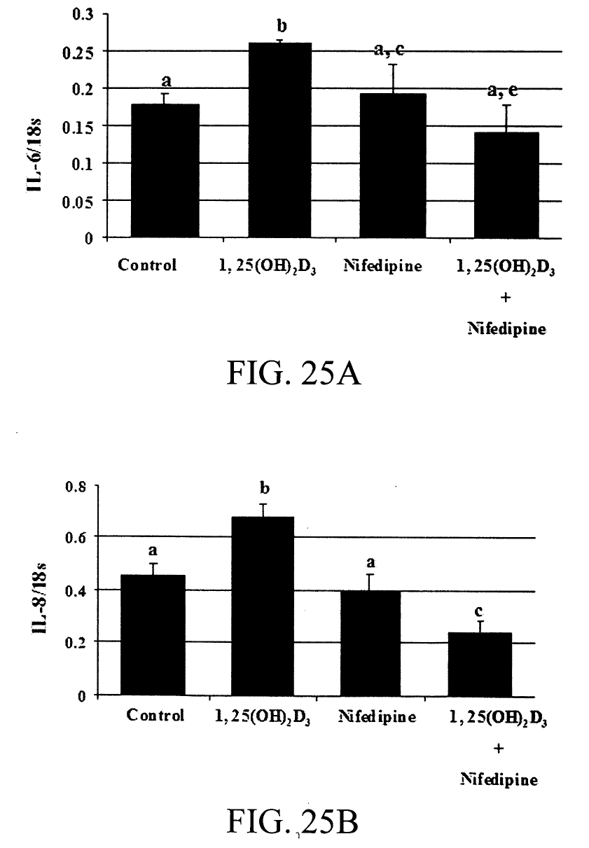

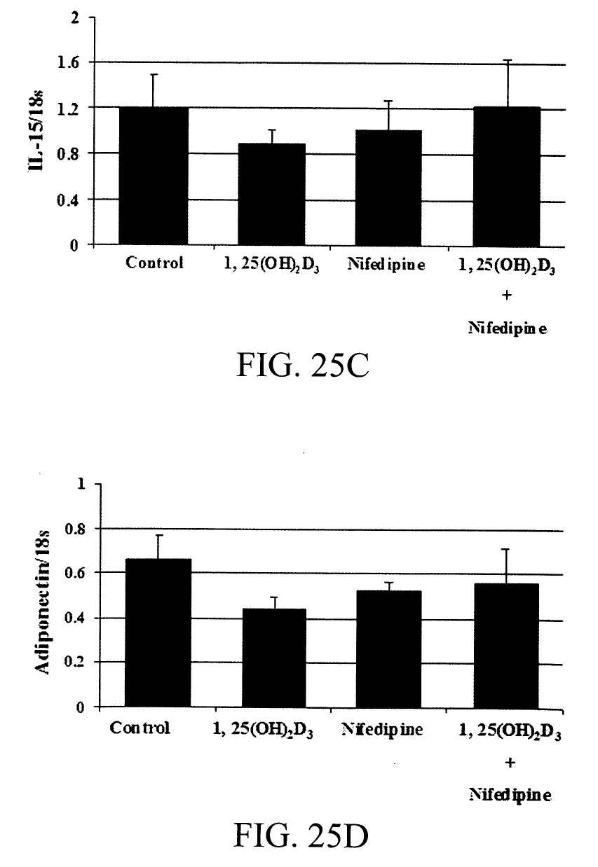

Similar results were also observed in differentiated human adipocytes; 1α, 25-(OH)₂-D₃ stimulated IL-6 and IL-8 expression by 53% and 49% respectively (FIG. 25A, p=0.004) (FIG. 25B, p<0.001), and the addition of nifedipine blocked this effect. However, we found no effect of 1α, 25-(OH)₂-D₃ or nifedipine on IL-15 (FIG. 25C, p=0.473) or adiponectin expression (FIG. 25D, p=0.377) in the human adipocytes.

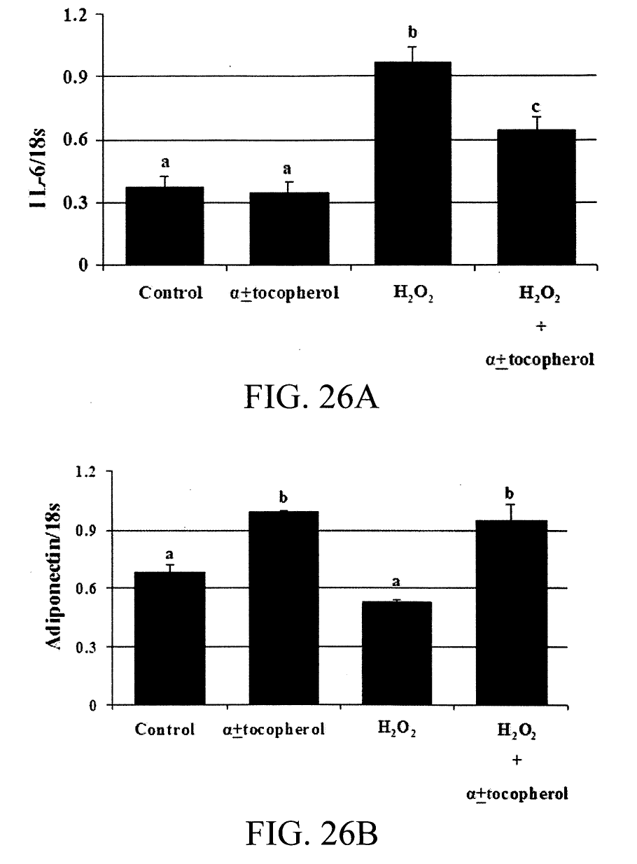

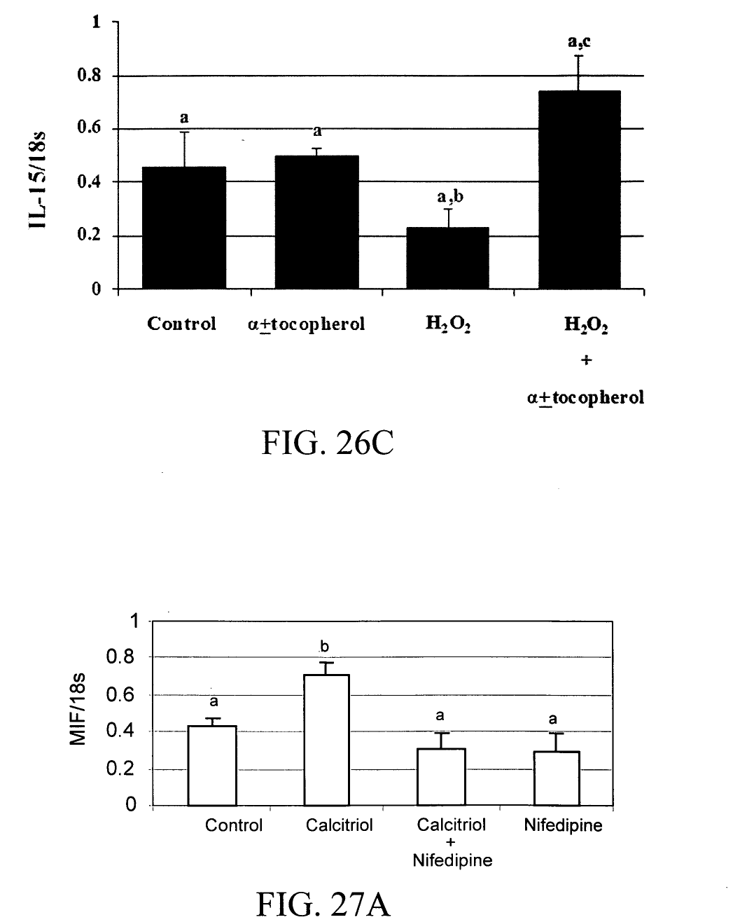

Reactive oxygen species exerted direct impact on cytokine production in cultured adipocytes. The direct role of ROS in regulation of adipose cytokine production was investigated in differentiated 3T3-L1 adipocytes. FIG. 26A shows that hydrogen peroxides increased IL-6 expression by 167% (p<0.001) and that this effect was attenuated by the addition of anti-oxidant α±tocopherol (p=0.016), indicating that ROS exerted a direct role in stimulation of inflammatory cytokine production. α±tocopherol also increased adiponectin production (p=0.002), although ROS (hydrogen peroxide) was without significant effect (p=0.06) (FIG. 26B). Similarly, there was no direct effect of ROS on IL-15 expression; however, addition of α±tocopherol markedly increased IL-15 by 2.2-fold as compared to H₂O₂-treated cells (P=0.043) (FIG. 26C), providing further evidence that oxidative stress is involved in adipocyte cytokine production

Discussion

Previous data from our laboratory demonstrate that dietary calcium exerts an anti-obesity effect and suppresses obesity associated oxidative stress via a 1α, 25-(OH)₂-D₃ mediated mechanism (Zemel, 2005b; Zemel, 2004). We have demonstrated that 1α, 25-(OH)₂-D₃ plays a direct role in the modulation of adipocyte Ca²⁺ signaling, resulting in an increased lipogenesis and decreased lipolysis (Shi et al., 2001). In addition, 1α, 25-(OH)₂-D₃ is also involved in regulation of metabolic efficiency by modulating adipocyte UCP2 expression (Shi et al., 2003). Accordingly, the suppression of 1α, 25-(OH)₂-D₃ by increasing dietary calcium attenuates adipocyte triglyceride accumulation and causes a net reduction in fat mass in both mice and humans in the absence of caloric restriction (Zemel et al., 2000; Zemel et al., 2005b), a marked augmentation of body weight and fat loss during energy restriction in both mice and humans (Zemel et al., 2000; Thompson et al., 2005; Zemel et al., 2004; Zemel et al., 2005a), and a reduction in the rate of weight and fat regain following energy restriction in mice (Sun et al., 2004a). Given that obesity and related disorders are associated with low grade systemic inflammation (Lee et al., 2005), it is possible that dietary calcium may also play a role in modulating adipose tissue cytokine production. Data from the present study demonstrate that dietary calcium decreased production of pro-inflammatory factors such as TNFα and IL-6 and increased anti-inflammatory molecules such as IL-15 and adiponectin in visceral fat. We also found that 1α, 25-(OH)₂-D₃ stimulated TNFα , IL-6 and IL-8 production in cultured human and murine adipocytes and that this effect was completely blocked by a calcium channel antagonist, suggesting that dietary calcium suppresses inflammation factor production in adipocyte and that 1α, 25-(OH)₂-D₃-induced Ca²⁺ influx may be a key mediator of this effect. FIGS. 22-23 demonstrate that dietary calcium decreased expression of pro-inflammatory factors (TNFα and IL-6) and increased anti-inflammatory molecules (IL-15 and adiponectin) in visceral adipose tissue and that dietary calcium up-regulates expression of IL-15 in both visceral adipose tissue and skeletal muscle, and stimulates adiponectin expression in visceral adipose tissue in aP2 agouti transgenic mice. This suggests that dietary calcium is involved in regulation of energy metabolism by modulating endocrine function of both adipose tissue and skeletal muscle, resulting in a pattern which favors reduced energy storage in adipose tissue and elevated protein synthesis and energy expenditure in skeletal muscle.

Obesity is associated with increased expression of inflammatory markers (Valle et al., 2005), while weight loss results in decreased expression and secretion of pro-inflammatory components in obese individuals (Clement et al., 2004). Accordingly, modulation of the adipose tissue mass appears to result in corresponding modulation of cytokine production. TNFα and IL-6 are two intensively studied cytokines in obesity and have been consistently found to be increased in the white adipose tissue of obese subjects (Cottam et al., 2004). Previous studies suggest that white adipose tissue contributes a considerable portion of circulating IL-6, with visceral fat contributing markedly more IL-6 compared with subcutaneous fat (Fried et al., 1998; Fain et al., 2004). Expression of TNFα is increased in inflammatory conditions such as obesity and cachexia and considered a likely mediator of insulin resistance associated with visceral adiposity (Hotamisligil et al., 1994; Ofei et al., 1996). Consistent with this, diet-induced obesity in present study resulted in increased expression of TNFA and IL-6 in visceral fat, and dietary calcium attenuated these effects.

IL-15 is highly expressed in skeletal muscle, where it exerts anabolic effects (Busquets et al., 2005). IL-15 administration reduces muscle protein degradation and inhibits skeletal muscle wasting in degenerative conditions such as cachexia (Carbo et al., 2000a). Interestingly, IL-15 exerts the opposite effect in adipose tissue; administration of IL-15 reduced fat deposition without altering food intake and suppressed fat gain in growing rats (Carbo et al., 2000b; Carbo et al., 2001). IL-15 also stimulates adiponectin secretion in cultured 3T3-L1 adipocytes (Quinn et al., 2005), indicating a role for IL-15 in regulating adipocyte metabolism. These observations suggest that IL-15 might be involved in a muscle-fat endocrine axis and regulate energy utilization between the two tissues (Argiles et al., 2005). We previously found calcium-rich diets to suppress fat gain and accelerate fat loss while protecting muscle mass in diet-induced obesity and during energy restriction, indicating that dietary calcium may similarly regulate energy partitioning in a tissue selective manner. In the present study, we provide the first in vivo evidence that dietary calcium up-regulates IL-15 expression in visceral adipose tissue and skeletal muscle, and stimulates adiponectin expression in visceral adipose tissue, skeletal muscle and stimulates adiponectin expression in visceral adipose tissue in aP2 agouti transgenic mice. This suggests that dietary calcium may also regulate energy metabolism, in part, by modulating these cytokines in both adipose tissue and skeletal muscle, thereby favoring elevated energy expenditure in adipose tissue and preserving energy storage in skeletal muscle. However, we found no effect of 1α, 25-(OH)₂-D₃ on IL-15 expression in human adipocytes. Since these human adipocytes were originally developed from subcutaneous fat, these results further support our in vivo observations of dietary calcium regulation of adipocyte cytokine production in a depot specific manner, although we do not have data from human visceral adipocytes for comparison.

We have recently shown that 1α, 25-(OH)₂-D₃ stimulated ROS production in cultured adipocytes and that suppression of 1α, 25-(OH)₂-D₃ via dietary calcium also attenuates adipose oxidative stress (Sun et al., 2006), suggesting a potential connection between oxidative tress and production of inflammatory factors. The present data demonstrate that hydrogen peroxide stimulates adipocyte IL-6 expression and α±tocopherol inhibits this effect. Although hydrogen peroxide showed no direct effect on the expression of anti-inflammatory factors adiponectin and IL-15, addition of α±tocopherol markedly elevated the expression of both, suggesting a direct role of oxidative stress in regulating inflammation. Indeed, previous studies have demonstrated that oxidative stress was augmented in adiposity, with ROS elevated in blood and tissue in various animal model of obesity (Suzuki et al., 2003; Furukawa et al., 2004), while markers of systemic oxidative stress were inversely related to plasma adiponectin in human subjects (Furukawa et al., 2004; Soares et al., 2005). Moreover, addition of oxidants suppressed expression of adiponectin and increased expression of IL-6, MCP-1 and PAI-1 (Soares et al., 2005). These results indicate that a local increase in oxidative stress in accumulated fat causes dysregulated production of adipocytokines. The role of adiposity in up-regulation of oxidative stress and inflammation has been investigated intensively. Fat accumulation stimulates NADPH oxidase expression in white adipose tissue (Sun et al., 2004d; Inoguchi et al., 2000). Further, NOX4, an isoform of NADPH oxidase, is expressed in adipocytes, but not in macrophage (Mahadev et al., 2004; Sorescu et al., 2002). Xu et al. (2003) and Weisberg et al. (2003) also reported that ROS stimulated macrophages infiltration of obese adipose tissue via ROS induced MCP-1 production and stimulated local NADPH oxidase expression and ROS production, indicating that both adipocytes and macrophages contribute to elevated oxidative stress in obesity.

Notably, the anti-inflammatory effect of dietary calcium is greater in visceral versus subcutaneous fat. We have previously observed similar pattern in adipocyte ROS production (Sun et al., 2006), in that ROS production and NADPH oxidase expression were markedly higher in visceral fat versus subcutaneous fat, suggesting that there may be an association between oxidative stress and inflammation in diet-induced obesity. Indeed, it was postulated that because visceral fat is more sensitive to lipolytic stimuli than adipose tissue stored at other sites, turnover of triacylglycerols and release of fatty acids into the portal circulation are increased (Wajchenberg, 2000). Free fatty acids, in addition, can stimulate ROS production by stimulating NADPH oxidase expression and activation (Soares et al., 2005). Accordingly, obesity associated with oxidative stress and inflammation may occur in a depot specific manner in adipose tissue, with significant higher ROS and inflammatory cytokines produced in visceral fat versus subcutaneous fat (Li et al., 2003). In summary, the present study demonstrates that dietary calcium suppresses obesity associated inflammatory status by modulating pro-inflammatory and anti-inflammatory factor expression, providing the evidence for the first time that increasing dietary calcium may contribute to suppression of obesity associated inflammation.

### EXAMPLE 4

### Calcium-Dependent Regulation of Macrophage Inhibitory Factor and CD14 Expression by Calcitriol in Human Adipocytes

Obesity increases oxidative stress and inflammatory cytokine production in adipose tissue, and our recent data demonstrate that dietary calcium attenuates obesity-induced oxidative stress and inflammation. This effect may be explained by dietary calcium inhibition of calcitriol, which we have shown to stimulate reactive oxygen species and inflammatory cytokine production in cultured adipocytes. However, adipose tissue includes both endothelial cells and leukocytes as well as adipocytes; these appear to contribute to a low-grade inflammatory state in obesity. Accordingly, the interaction between adipocytes and leukocytes may play an important role in the local modulation of inflammation. Consequently, we investigated calcitriol modulation of the expression of macrophage inhibitory factor (MIF) and macrophage surface specific protein CD14, two key factors in regulating macrophage function and survival, in differentiated human adipocytes. Calcitriol markedly increased MIF and CD 14 expression by 59% (p=0.001) and 33% (p=0.008) respectively, while calcium channel antagonism with nifedipine completely reversed these effects, indicating that calcitriol stimulates MIF and CD 14 expression via a calcium-dependent mechanism. Similar results were also found in cultured 3T3-L1 adipocytes; in addition, calcitriol also up-regulated M-CSF, MIP, MCP-1 (monocyte chemoattractant protein-1) and IL-6 expression in 3T3-L1 adipocyte and stimulated tumor necrosis factor-α (TNF-α) and IL-6 expression in RAW264 macrophage cultured alone and this effect was blocked by either a calcium channel antagonist (nifedipine) or a mitochondrial uncoupler (DNP). Moreover, co-culture of 3T3-L1 adipocytes with RAW 264 macrophages significantly increased the expression and production of multiple inflammatory cytokines in response to calcitriol in both cell types. These data suggest that calcitriol may regulate macrophage activity by modulating adipocyte production of factors associated with macrophage function. These data also provide additional explanation for our recent observations that suppression of calcitriol by dietary calcium decreases obesity associated oxidative stress and inflammation.

Materials and Methods

Cell culture: Human preadipocytes used in this study were supplied by Zen-Bio (Research Triangle, N.C.). Preadipocytes were inoculated in DMEM/Ham's F-10 medium (DMEM-F10) (1:1, vol/vol) containing 10% FBS, 15 mmol/L HEPES, and antibiotics at a density of 30,000 cells/cm². Confluent monolayers of preadipocytes were induced to differentiate with a standard differentiation medium consisting of DMEM-F 10 (1:1, vol/vol) medium supplemented with 15 mmol/L HEPES, 3% FBS, 33 μmol/L biotin, 17 μmol/L pantothenate, 100 nmol/L insulin, 0.25 μmol/L methylisobutylxanthine, 1 μmol/L dexamethasone, 1 μmol/L BRL49653, and antibiotics. Preadipocytes were maintained in this differentiation medium for 3 days and subsequently cultured in adipocyte medium in which BRL49653 and MIX were omitted. Cultures were re-fed every 2-3 days.

RAW 264 macrophages and 3T3-L1 preadipocytes (American Type Culture Collection) were incubated at a density of 8000 cells/cm2 (10 cm2 dish) and grown in Dulbecco's modified Eagle's medium (DMEM) containing 10% FBS and antibiotics (adipocyte medium) at 37° C. in 5% CO2 in air. Confluent 3T3-L1 preadipocytes were induced to differentiate with a standard differentiation medium consisting of DMEM-F10 (1:1, vol/vol) medium supplemented with 1% FBS, 1 μM dexamethasone, IBMX (0.5 mM) and antibiotics (1% Penicillin-Streptomycin). Preadipocytes were maintained in this differentiation medium for 3 days and subsequently cultured in adipocyte medium. Cultures were re-fed every 2-3 days to allow 90% cells to reach fully differentiation for 3T3-L1 adipocytes or grow to a confluence for RAW 264 before conducting chemical treatment. Cells were treated with or without calcitriol (10 nmol/L) GDP (100 μmol/L) and/or nifedipine (10 μmol/L) for 48 hours, as indicated in each figure.

Cells were washed with fresh adipocyte medium, re-fed with medium containing the indicated treatments, and incubated at 37° C. in 5% CO₂ for 48 hours before analysis. Cell viability was measured via trypan blue exclusion.

Cell Culture

Human adipocytes (Zen-Bio, Inc.), 3T3-L1 adipocytes, RAW264 macrophages were obtained and co-cultured by using transwell inserts with 0.4 μm porous membranes (Corning) to separate adipocytes and macrophages. All data are expressed as mean±SEM. Data were evaluated for statistical significance by analysis of one-way or two-way variance (ANOVA; means with different letter differ, p<0.05).

Total RNA Extraction:

A total cellular RNA isolation kit (Ambion, Austin, Tex.) was used to extract total RNA from cells according to manufacturer's instruction. The concentration and purity of the isolated RNA was measured spectrophotometrically and the integrity of RNA sample was analyzed by BioAnalyzer (Agilent 2100, Agilent Tenchnologies).

Quantitative Real Time PCR:

Adipocyte and muscle 18s, CD14, TNFα, MIP, M-CSF, IL-6 and MCP-1 were quantitatively measured using a Smart Cycler Real Time PCR System (Cepheid, Sunnyvale, Calif.) with a TaqMan 1000 Core Reagent Kit (Applied Biosystems, Branchburg, N.J.). The primers and probe sets were obtained from Applied Biosystems TaqMan® Assays-on-Demand™ Gene Expression primers and probe set collection according to manufacture's instruction. Pooled adipocyte total RNA was serial-diluted in the range of 1.5625-25 ng and used to establish a standard curve; total RNAs for unknown samples were also diluted in this range. Reactions of quantitative RT-PCR for standards and unknown samples were also performed according to the instructions of Smart Cycler System (Cepheid, Sunnyvale, Calif.) and TaqMan Real Time PCR Core Kit (Applied Biosystems, Branchburg, N.J.). The mRNA quantitation for each sample was further normalized using the corresponding 18s quantitation.

Cytokine Antibody Array:

A TansSignal™ mouse cytokine antibody array kit (Panomics, Fremont, Calif.) was used to detect cytokine protein released in culture medium according to the manufacture's instruction. Briefly, membranes immobilized with capture antibodies specific to particular cytokine proteins was incubated with 1× blocking buffer for 2 hours and then blocking buffer was washed three times using washing buffer. Then, membranes were incubated in samples for 2 hours to allow cytokine protein in the culture medium to bind to the capture antibody on the membrane. At the end of the incubation, unbound protein was washed away using washing buffer. The membranes were then incubated with biotin-conjugated antibody mix which binds to a second epitope on the protein. The membrane was then washed and incubated with strepavidin-HRP to visualize the antibody-protein complexes on the array to determine which cytokines are present in the sample via chemiluminescent signal which was detected using X-ray film.

Statistical Analysis:

Each treatment was replicated with n=6, and data are expressed as mean±SEM. Data were evaluated for statistical significance by analysis of variance (ANOVA) and significantly different group means were then separated by the least significant difference test by using SPSS (SPSS Inc, Chicago, Ill.). The co-culture experiments were analyzed via two-way (treatment X culture condition) ANOVA.

Results and Discussion

Obesity is characterized by increased oxidative and inflammatory stress. Adipose tissue is a significant source of reactive oxygen species (ROS) and expresses and secretes a wide variety of pro-inflammatory components in obese individuals, such as TNF-α and IL-6. Notably, the adipose tissue is not only composed of adipocytes but also contains a stromal vascular fraction that includes blood cells, endothelial cells and macrophages. Although adipocytes directly generate inflammatory mediators, adipose tissue-derived cytokines also originate substantially from non-fat cells, among which infiltrated macrophages appear to play a prominent role. Infiltration and differentiation of adipose tissue-resident macrophages are under the local control of chemokines, many of which are produced by adipocytes. Accordingly, the cross-talk between adipocytes and macrophages may be a key factor in mediating inflammatory and oxidative changes in obesity.

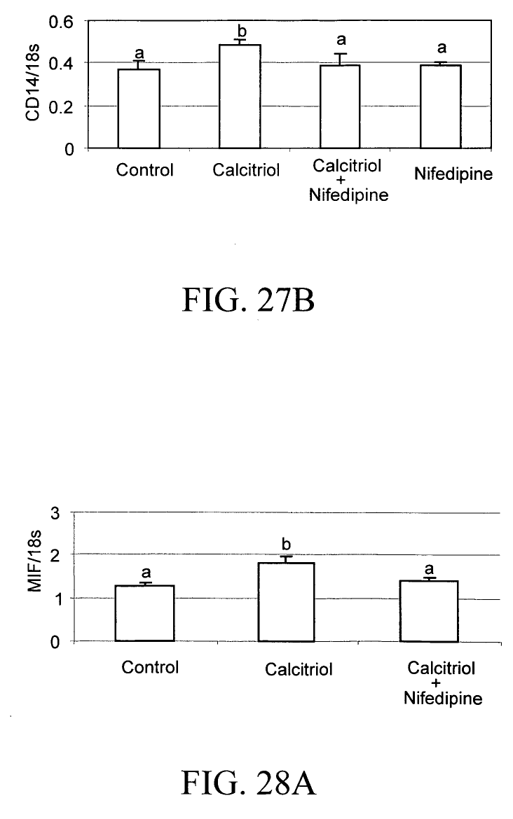

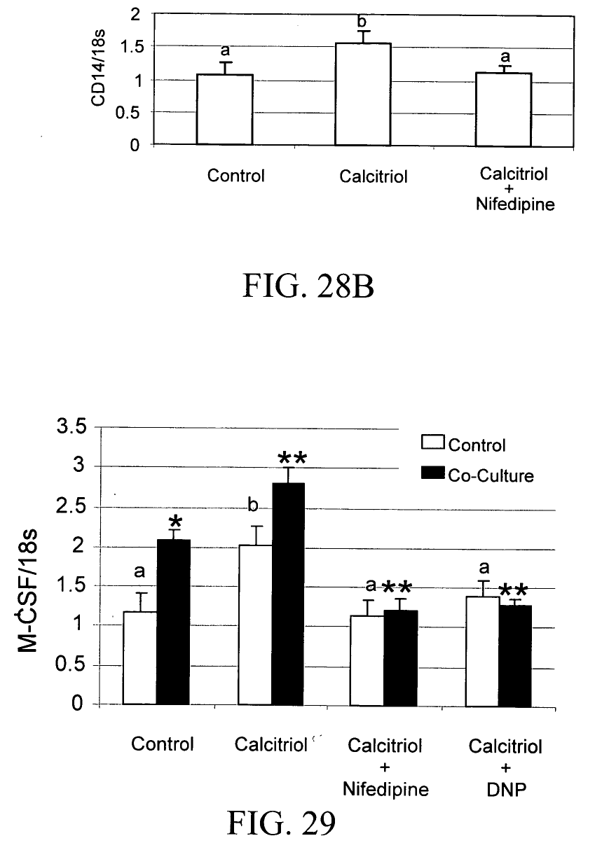

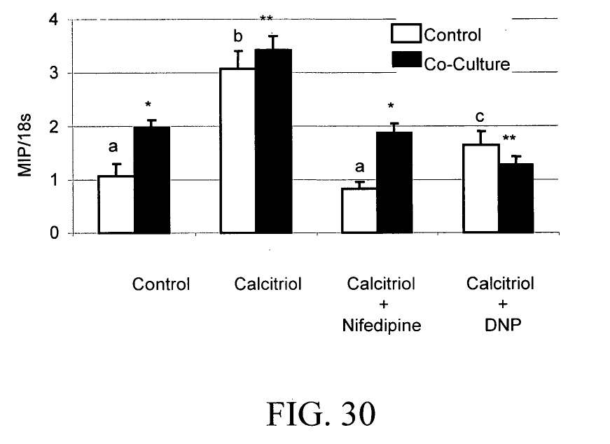

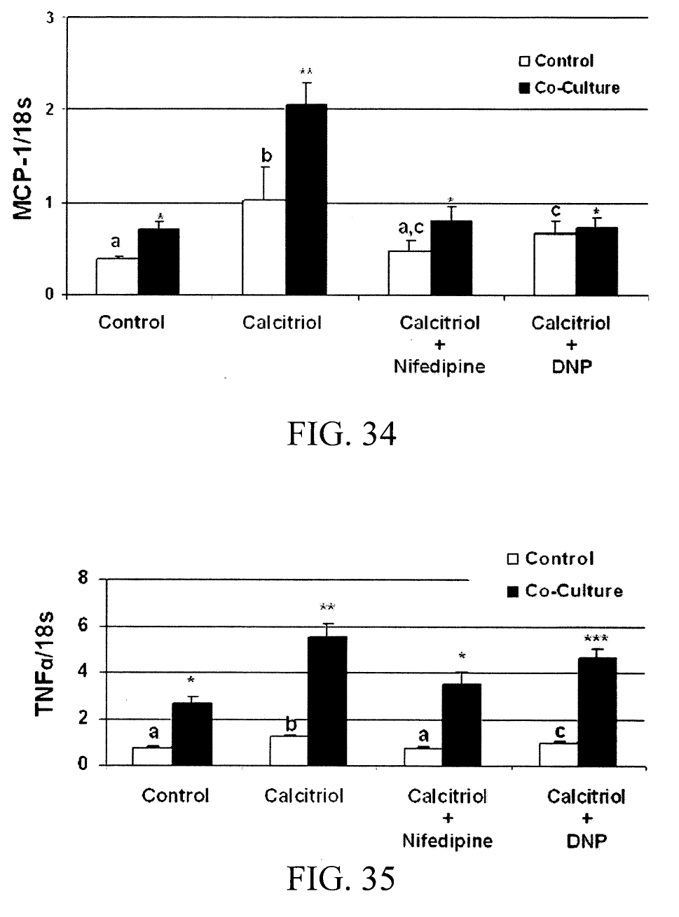

FIG. 27 demonstrates that calcitriol increased MIF (FIG. 27A) and CD14 (FIG. 27B) expression in human adipocytes by 59% and 33% respectively, and addition of a calcium channel antagonist (nifedipine) reversed this effect, indicating a role of intracellular calcium in mediating this effect. FIG. 28, consistent with FIG. 27, demonstrates that calcitriol increased MIF expression by 50% (FIG. 28A) and CD14 expression by 45% (FIG. 28B) in mouse (3T3-L1) adipocytes and the addition of a calcium channel antagonist (nifedipine) reversed this effect. FIGS. 29, 30 and 31 show that calcitriol markedly stimulate inflammatory cytokines M-CSF (FIG. 29), MIP (FIG. 30), IL-6 (FIG. 31) and MCP-1 (FIG. 34) expression in 3T3-L1 adipocytes, and co-culture with RAW 264 macrophages enhance this effect, indicating a potential role of adipocytes in regulation of local resident macrophages activity and that calcitriol may regulate macrophage activity by modulating adipocyte production of factors associated with macrophage function. Main effects of chemical treatment and culture status were significant (p<0.02).

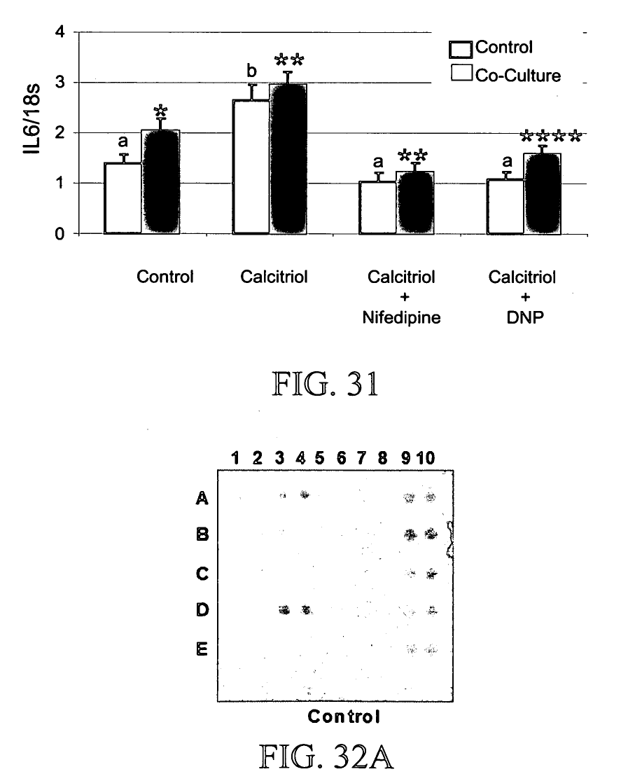

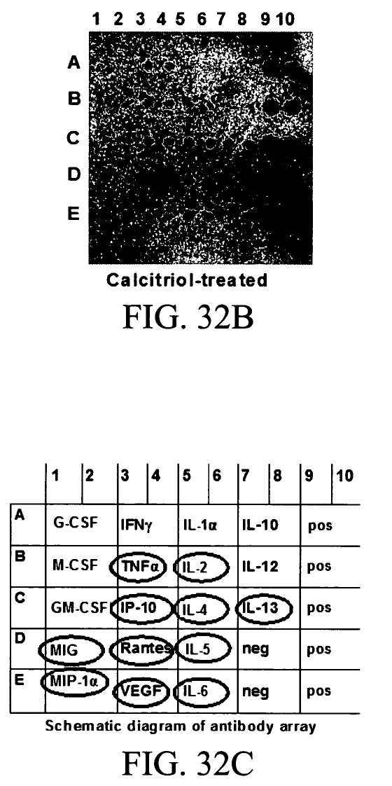

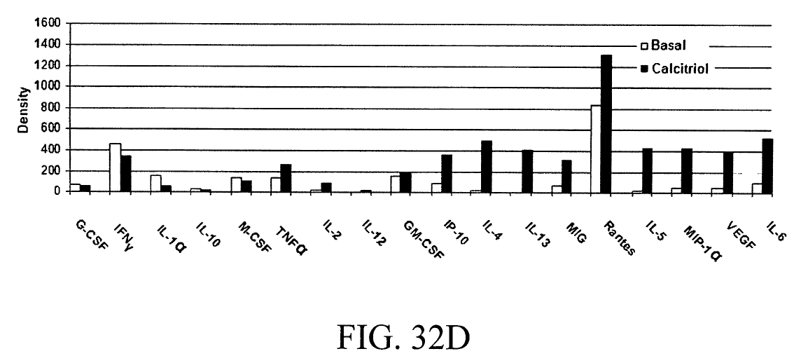

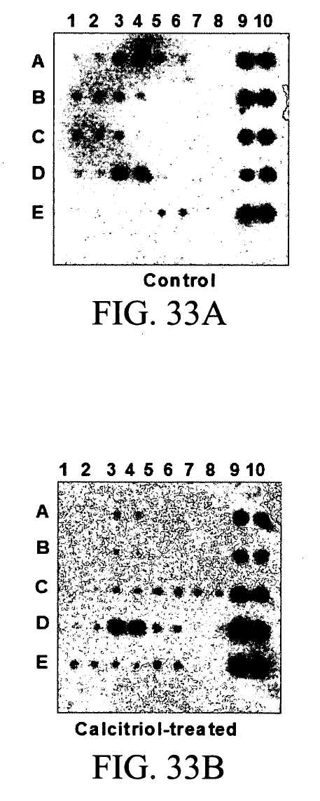

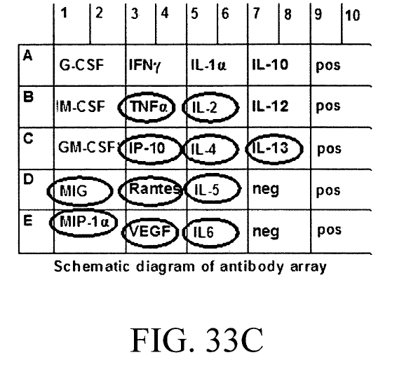

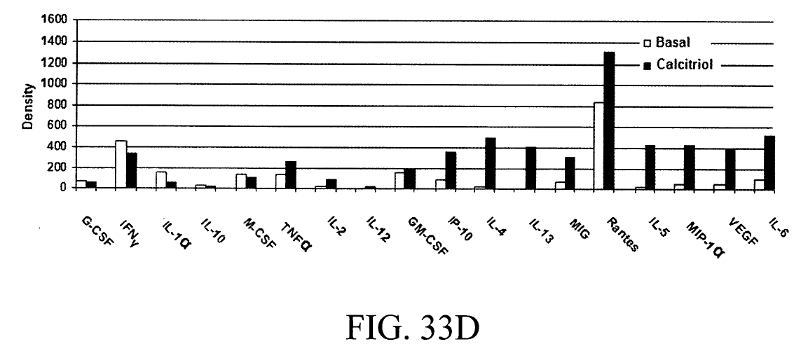

A cytokine antibody array was used to further investigate the effects of calcitriol on release of major inflammatory cytokines from adipocytes. These protein data support the gene expression observations, as calcitriol up-regulated production of multiple inflammatory cytokine proteins in differentiated 3T3-L1 adipocytes cultured alone (FIG. 32); these include TNFA, IL-6, IL-2, Granulocyte/Macrophage-Colony Stimulating Factor (GM-CSF), Interferon-inducible protein-10 (IP-10), IL-4, IL-13, macrophage induced gene (MIG), regulated upon T cell activation expressed secreted (RANTES), IL-5, macrophage inflammatory protein la (MIP-la) and vascular endothelial growth factor (VEGF). Co-culture of 3T3-L1 adipocytes with macrophages significantly up-regulated production of cytokines such as interferon γ (IFN γ), TNFα, G-CSF and MIP-1α compared with 3T3-L1 cultured alone (FIG. 33), and calcitriol further stimulated inflammatory cytokine production (FIG. 33).

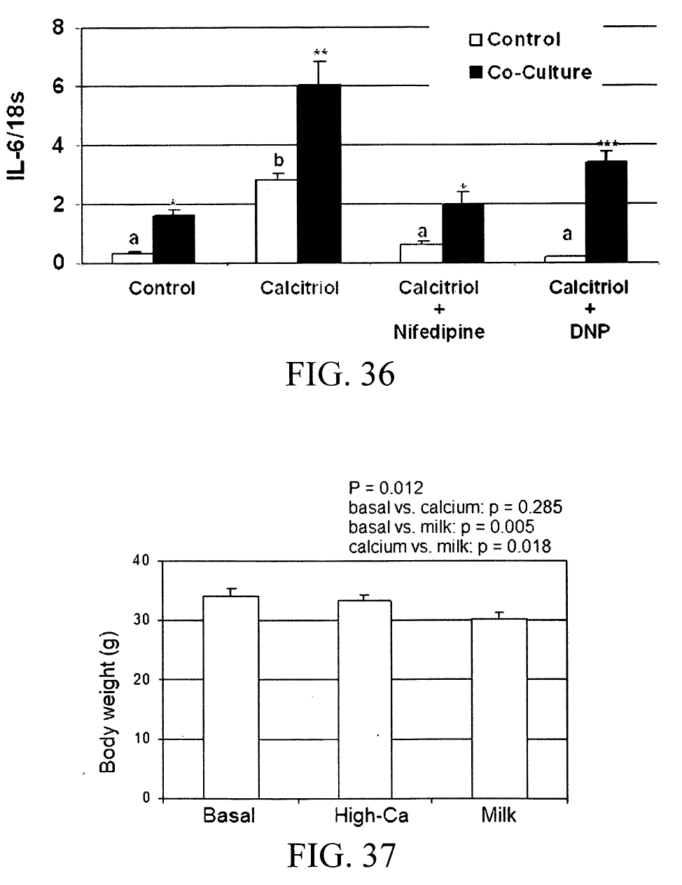

Calcitriol also markedly stimulated TNFα expression by 91% (FIG. 35) and IL-6 by 796% (FIG. 36) in RAW 264 macrophages cultured alone and these effects were blocked by adding nifedipine or DNP. Co-culture of macrophages with differentiated 3T3-L1 adipocytes markedly augmented TNFα (FIG. 35) and IL-6 (FIG. 36) expression in macrophages, and these effects were further enhanced by calcitriol.

Data from this study demonstrate that calcitriol stimulates production of adipokines associated with macrophage function and increases inflammatory cytokine expression in both macrophages and adipocytes; these include CD14, MIF, M-CSF, MIP, TNFα, IL-6 and MCP-1 in adipocytes, and TNFα and IL-6 in macrophages. Consistent with this, the cytokine protein array identified multiple additional inflammatory cytokines which were up-regulated by calcitriol in adipocytes. Moreover, calcitriol also regulated cross-talk between macrophages and adipocytes, as shown by augmentation of expression and production of inflammatory cytokines from adipocytes and macrophages in coculture versus individual culture. These effects were attenuated by either calcium channel antagonism or mitochondrial uncoupling, indicating that the pro-inflammatory effect of calcitriol are mediated by calcitriol-induced stimulation of Ca²⁺ signaling and attenuation of mitochondrial uncoupling.

These data demonstrate that calcitriol regulates both adipocyte and macrophage production of inflammatory factors via calcium-dependent and mitochondrial uncoupling-dependent mechanisms and that these effects are amplified with co-culture of both cell types. These data further suggest that strategies for reducing circulating calcitriol levels, such as increasing dietary calcium, may regulating adipocyte macrophage interaction and thereby attenuate local inflammation in adipose tissue.

### EXAMPLE 5

### Dietary Calcium and Dairy Modulation of Oxidative and Inflammatory Stress in Mice

Obesity is associated with subclinical chronic inflammation which contributes to obesity-associated co-morbidities. Calcitriol (1, 25-(OH)₂-D₃) regulates adipocyte lipid metabolism, while dietary calcium inhibits obesity by suppression of calcitriol. We have recently shown this anti-obesity effect to be associated with decreased oxidative and inflammatory stress in adipose tissue in vivo. However, dairy contains additional bioactive compounds which markedly enhance its anti-obesity activity and which we propose will also enhance its ability to suppress oxidative and inflammatory stress. Accordingly, the objective of this study was to determine the effects of dietary calcium and dairy on oxidative and inflammatory stress in a mouse model (aP2-agouti transgenic mice) that we have previously demonstrated to be highly predictive of the effects of calcium and dairy on adiposity in humans and have recently established as a model for the study of oxidative stress.

Study: Six-week old aP2-agouti transgenic mice were fed a modified AIN 93-G diet with sucrose as the sole carbohydrate source (64% of energy), and fat increased to 25% of energy with lard. A total of 30 animals will be studied for three weeks (n=10/group), as follows: Control (low Ca) suboptimal calcium (0.4%); High Ca with 1.2% calcium in the form of CaCO₃; High Dairy: 50% of the protein was replaced by nonfat dry milk and dietary calcium will be increased to 1.2% Approximately ½ of the additional calcium was derived from the milk and the remainder was added as CaCO₃. Food intake and spillage was monitored daily and body weight and blood glucose was measured weekly. Following three weeks of feeding, all animals from each group were killed for determination of the following outcome measurements: plasma insulin, MDA calcitriol and cytokine (IL-6, MCP, IL-15, adiponectin and TNF-α; adipose Tissue:IL-6, MCP, IL-15, adiponectin, TNF-α and NADPH oxidase expression, tissue release of adipokines, ROS production; muscle: real-time PCR of NADPH oxidase, IL-6 and IL-15; Tissue release of cytokines, ROS production.

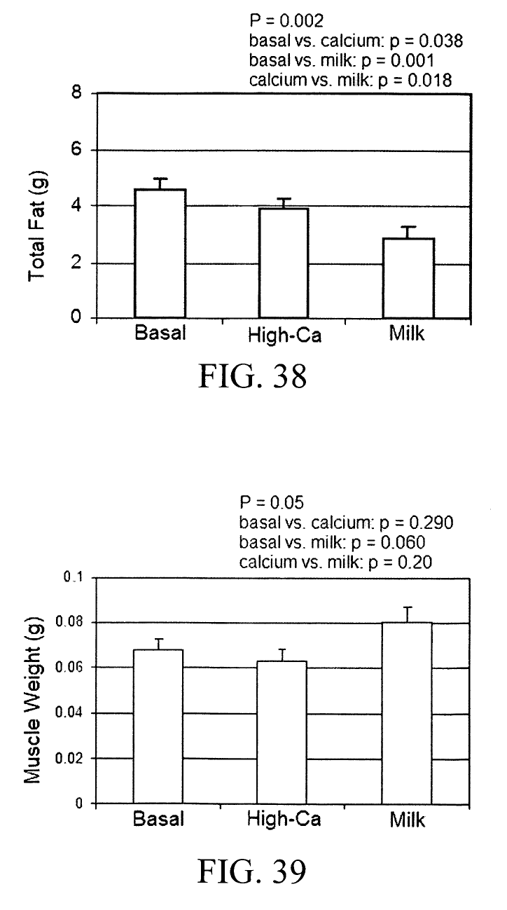

Results: Body weight and composition: A three-week study duration was utilized in order to avoid major calcium- and milk-induced alterations in adiposity, as adiposity-induced oxidative stress could cause a degree of confounding. Nonetheless, there were modest, but statistically significant diet-induced changes in body weight and composition. The high calcium diet was without effect on body weight, but the milk diet did induce a significant decrease in total body weight (FIG. 37). In contrast, both the calcium and the milk diets caused significant decreases in body fat, with the milk diet eliciting a significantly greater effect (FIG. 38).

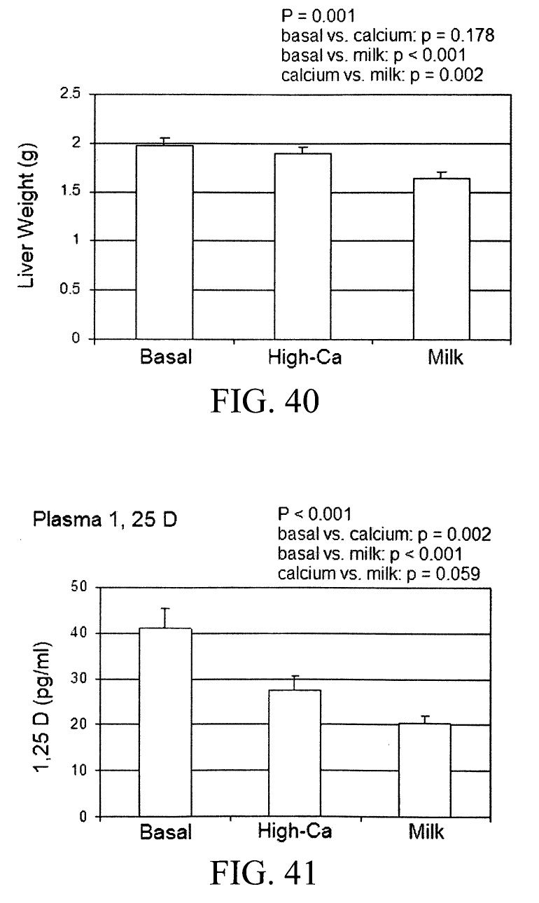

Skeletal muscle weight (soleus+gastrocnemius) exhibited overall differences (p=0.05) among the dietary groups. The milk group had significantly greater skeletal muscle mass than the calcium group (p=0.02) and a tendency towards greater skeletal muscle mass than the basal group (p=0.06) (FIG. 39). Liver weight was slightly, but significantly, reduced by the milk diet (FIG. 40).

Circulating calcitriol: The high calcium diet caused a reduction in plasma 1, 25-(OH)₂-D (calcitriol) (p=0.002), and there was a trend (p=0.059) towards a further decrease in plasma calcitriol on the high milk diet (FIG. 41). The reason for the difference between the calcium and milk diets in suppressing calcitriol is not clear, as they contain the same levels of dietary calcium.

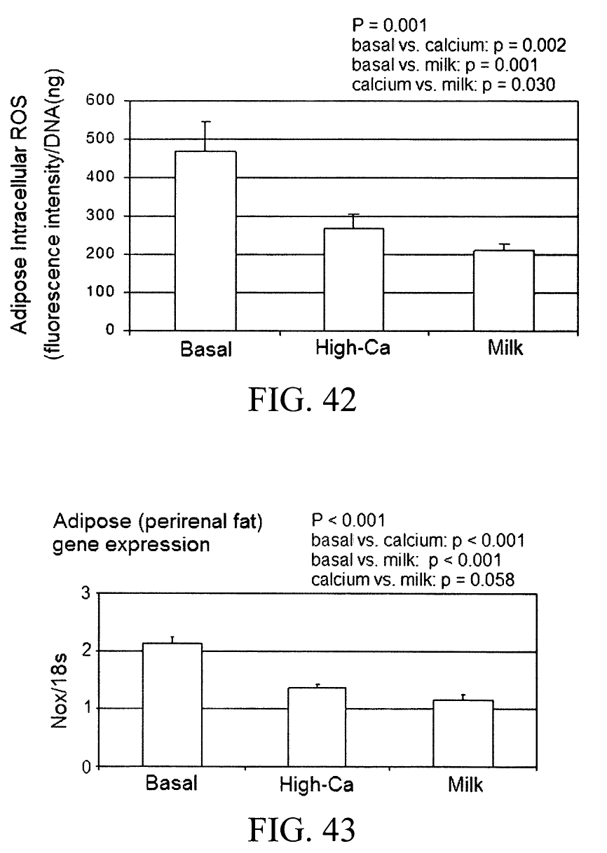

Reactive Oxygen Species and Oxidative Stress: Adipose tissue reactive oxygen species (ROS) production was significantly reduced by the high calcium diet (p=0.002), consistent with our previous data, and further reduced by the milk diet (p=0.03) (FIG. 42). Consistent with this, the high calcium diet caused a significant reduction in adipose tissue NADPH oxidase (Nox; one of the sources of intracellular ROS) expression (p=0.001) and there was a strong trend (p=0.056) towards a further suppression of NOX on the milk diet (FIG. 43).

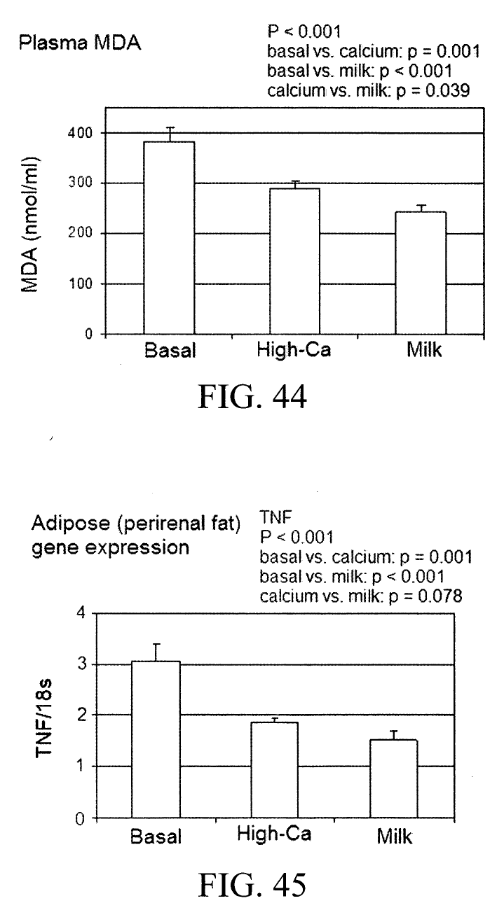

These changes were reflected in significant decreases in systemic lipid peroxidation, as demonstrated by significant decreases in plasma malonaldehyde (MDA). Plasma MDA was significantly decreased by both the calcium and milk diets (p=0.001), with a significantly greater effect of the milk diet (p=0.039) (FIG. 44).

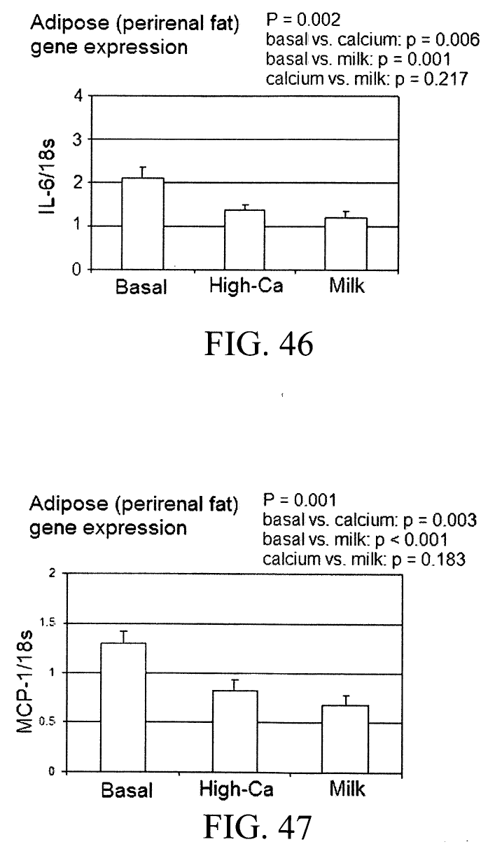

Inflammatory Stress: In general, the high calcium diet resulted in suppression of inflammatory markers and an upregulation of anti-inflammatory markers, and the milk diet exerted a greater effect than the high calcium diet. Adipose tissue expression of TNF-α (FIG. 45), IL-6 (FIG. 46) and MCP (FIG. 47) were all significantly suppressed by the high calcium diet. Expression of each of these inflammatory cytokines was lower on the milk diet than on the high calcium diet, but this difference was only statistically evident as a trend for TNF-α (p=0.076).

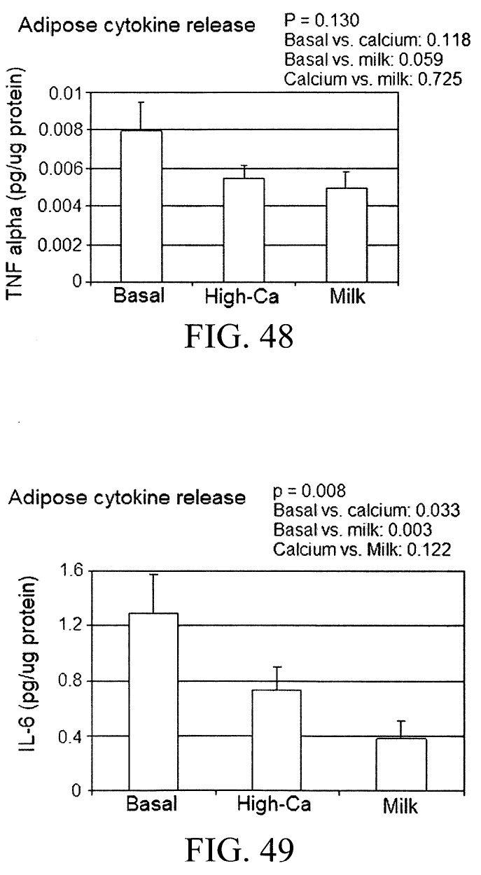

Consistent with these data, the calcium and milk diets caused significant reductions in the release of inflammatory cytokines (TNF-α, FIG. 48; IL6, FIG. 49) from adipose tissue. There was trend towards a greater effect of the milk vs. calcium diet, but this difference was not statistically significant.

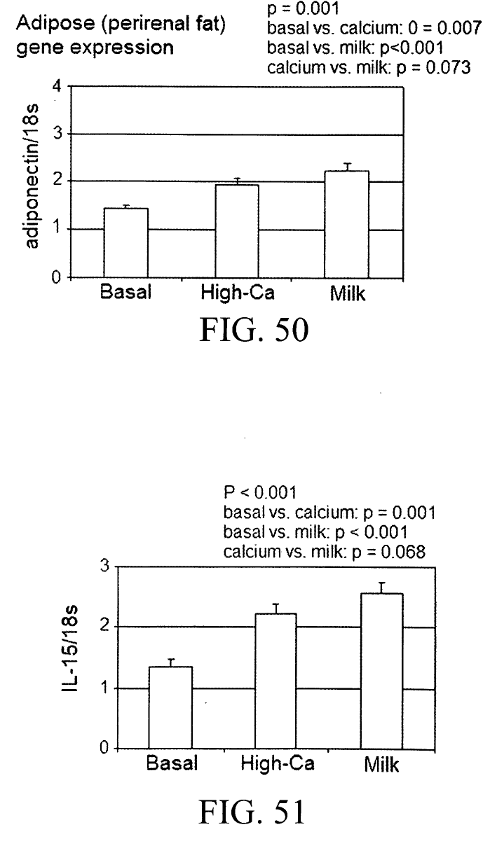

There was a corresponding up-regulation of adipose tissue anti-inflammatory cytokine expression on the high calcium diets. The high calcium and milk diets increased adiponectin expression (p=0.001; FIG. 50) and IL-15 expression (p=0.001; FIG. 51), and there was a trend for a further increase on the milk diet vs. high calcium diet (p=0.073 for adiponectin; p=0.068 for IL-15).

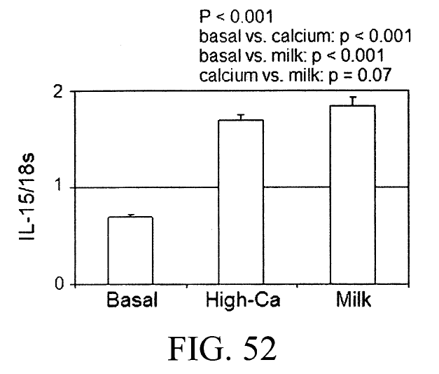

Similarly, there was a marked increase in skeletal muscle IL-15 expression on the high calcium diet (p<0.001), with a further increase on the milk diet (p=0.07; FIG. 52).

These data clearly demonstrate that dietary calcium suppresses both adipose tissue and systemic oxidative stress, and that dairy (milk) exerts a significantly greater effect. It may be argued that the reduced adipose tissue mass may have contributed to the decrease in oxidative stress on the high milk diet. However, this is unlikely, as the decrease in adiposity was quite modest compared to the decrease in oxidative stress. Moreover, the decrease in adipose tissue ROS production and Nox expression are normalized to reflect decreases per adipocyte as well as total systemic decreases. Accordingly, these decreases in oxidative stress appear to be direct effects of the high calcium and high dairy diets. Data from this study also demonstrate a marked reduction in adipose tissue-derived inflammatory cytokines on the high calcium diets, with a strong trend towards further suppression of inflammatory cytokines on the milk vs. high calcium diet. Moreover, anti-inflammatory cytokine expression is significantly up-regulated on the high calcium diet, with further improvements evident on the milk vs. calcium diet. Although there are additional analyses to be completed, these data indicate a marked shift in the ratio of anti-inflammatory to inflammatory cytokines on high calcium diets, with further improvements in this ratio when milk is used as the calcium source. Thus, data from this pilot study strongly suggest that dietary calcium suppresses oxidative and inflammatory stress, consistent with our previous data, and that other components of milk enhance this effect to produce greater control of both oxidative and inflammatory stress.

TABLE 1
Body weight and fat pad weights at baseline and 3-week after diet treatment
in aP2-agouti transgenic mice fed low and high calcium diets¹.

| | Baseline | | 3-week after | | |
|---|---|---|---|---|---|
| | Low-Ca diet | High-Ca diet | Low-Ca diet | High-Ca diet | p value |
| Body weight (g)¹ | 25.28 ± 0.39 | 24.47 ± 0.47 | 32.96 ± 0.95 | 28.56 ± 0.57* | P = 0.023 |
| Body fat (g) | N/A | N/A | 4.47 ± 0.37 | 2.44 ± 0.23* | P = 0.007 |
| Subcutaneous fat² (g) | N/A | N/A | 1.76 ± 0.17 | 0.94 ± 0.11* | P = 0.015 |
| Visceral fat³ (g) | N/A | N/A | 2.48 ± 0.19 | 1.31 ± 0.11* | P = 0.004 |

¹Values are means ± SD, n = 10. p-values indicate significant level between animals on the basal diet and those on the high-Ca diet.
²Subscapular fat pad
³Sum of perirenal and abdominal fat pads

### REFERENCES

Agarwal, A. K. (2003) “Cortisol metabolism and visceral obesity: role of 11beta-hydroxysteroid dehydrogenase type I enzyme and reduced co-factor NADPH” Endocr Res. 29:411-418.
Alwine, J. C. et al. (1977) “Method for detection of specific RNAs in agarose gels by transfer to diazobenzyloxymethyl-paper and hybridization with DNA probes” Proc. Natl. Acad. Sci. USA 74:5350-5354.
Amstad, P. A., Krupitza, G., Cerutti, P. A. (1992) “Mechanism of c-fos induction by active oxygen” Cancer Res. 52:3952-60.
Argiles, J. M., Lopez-Soriano, J., Almendro, V., Busquets, S., Lopez-Soriano, F. J. (2005) “Cross-talk between skeletal muscle and adipose tissue: a link with obesity” Med Res Rev. 25:49-65.
Atabek, M. E., Vatansev, H., Erkul, I. (2004) “Oxidative stress in childhood obesity” J Pediatr Endocrinol Metab 17:1063-1068.
Banakar, M. C., Paramasivan, S. K., Chattopadhyay, M. B., Datta, S., Chakraborty, P., Chatterjee, M., Kannan, K., Thygarajan, E. (2004) “1alpha, 25-dihydroxyvitamin D3 prevents DNA damage and restores antioxidant enzymes in rat hepatocarcinogenesis induced by diethylnitrosamine and promoted by Phenobarbital” World J Gastroenterol 10:1268-75.
Berchtold, M. W. et al. (1989) “A simple method for direct cloning and sequencing cDNA by the use of a single specific oligonucleotide and oligo(dT) in a polymerase chain reaction (PCR)” Nuc. Acids. Res. 17:453.
Brand, M. D., Buckingham, J. A., Esteves, T. C., Green, K., Lambert, A. J., Miwa, S., Murphy, M. P., Pakay J. L., Talbot, D. A., Echtay, K. S. (2004) “Mitochondrial superoxide and aging: uncoupling-protein activity and superoxide production” Biochem Soc Symp 71:203-213.
Brookes, P. S. (2005) “Mitochondrial H(+) leak and ROS generation: an odd couple” Free Radic Biol Med 38:12-23.
Busquets, S., Figueras, M. T., Meijsing, S., Carbo, N., Quinn, L. S., Almendro, V., Argiles, J. M., Lopez-Soriano, F. J. (2005) “Interleukin-15 decreases proteolysis in skeletal muscle: a direct effect” Int J Mol Med. 16:471-6.
Carbo, N., Lopez-Soriano, J., Costelli, P., Busquets, S., Alvarez, B., Baccino, F. M., Quinn, L. S., Lopez-Soriano, F. J., Argiles, J. M. (2000a) “Interleukin-15 antagonizes muscle protein waste in tumour-bearing rats” Br J Cancer 83:526-531.
Carbó, N., J. Lopez-Soriano, P. Costelli, S. Busquets, B. Alvarez and F. M. Baccino et al. (2000b) “Interleukin-15 antagonizes muscle protein waste in tumour-bearing rats” Br J Cancer 83:526-531.
Carbó, N. J. Lopez-Soriano, P. Costelli, B. Alvarez, S. Busquets and F. M. Baccino et al. (2001) “Interleukin-15 mediates reciprocal regulation of adipose tissue and muscle mass: a potential role in body weight control” Biochim Biophys Acta 1526:17-24.
Cerutti, P., Ghosh, R., Oya, Y., Amstad, P. (1994) “The role of the cellular antioxidant defense in oxidant carcinogenesis” Environ Health Perspect 102:123-9.
Chang, L., Karin, M. (2001) “Mammalian MAP kinase signalling cascades” Nature 410:37-40.
Chung, S. S., Ho, E. C., Lam, K. S., Chung, S. K. (2003) “Contribution of polyol pathway to diabetes-induced oxidative stress” J Am Soc Nephrol 14:S233-S236.
Clement, K., Viguerie, N., Poitou, C., Carette, C., Pelloux, V., Curat, C. A., Sicard, A., Rome, S., Benis, A., Zucker, J. D., Vidal, H., Laville, M., Barsh, G. S., Basdevant, A., Stich, V., Cancello, R., Langin, D. (2004) “Weight loss regulates inflammation-related genes in white adipose tissue of obese subjects” FASEB J 18:1657-1669.
Cottam, D. R., S. G. Mattar, E. Barinas-Mitchell, G. Eid, L. Kuller and D. E. Kelley et al. (2004) “The chronic inflammatory hypothesis for the morbidity associated with morbid obesity: implications and effects of weight loss” Obes Surg 14:589-600.
Ermak, G., Davies, K. J. (2002) “Calcium and oxidative stress: from cell signaling to cell death” Mol Immunol 38:713-721.
Fain, J. N., A. K. Madan, M. L. Hiler, P. Cheema and S. W. Bahouth (2004) “Comparison of the release of adipokines by adipose tissue, adipose tissue matrix, and adipocytes from visceral and subcutaneous abdominal adipose tissues of obese humans” Endocrinology 145:2273-82.
Furukawa, S., Fujita, T., Shimabukuro, M., Iwaki, M., Yamada, Y., Nakajima, Y., Nakayama, O., Makishima, M., Matsuda, M., Shimomura, I. (2004) “Increased oxidative stress in obesity and its impact on metabolic syndrome” J Clin Invest. 114:1752-1761.
Fried, S. K., D. A. Bunkin and A. S. Greenberg (1998) “Omental and subcutaneous adipose tissues of obese subjects release interleukin-6: depot difference and regulation by glucocorticoid” J Clin Endocrinol Metab 83:847-50.
Giri, D. K., Aggarwal, B. B. (1998) “Constitutive activation of NF-kappaB causes resistance to apoptosis in human cutaneous T cell lymphoma HuT-78 cells. Autocrine role of tumor necrosis factor and reactive oxygen intermediates” J Biol Chem. 273:14008-14.
Gordeeva, A. V., Zvyagilskaya, R. A., Labas, Y. A. (2003) “Cross-talk between reactive oxygen species and calcium in living cells” Biochemistry (Mosc). 68:1077-1080.
Hollander, M. C., Fornace, A. J. Jr. (1989) “Induction of fos RNA by DNA-damaging agents” Cancer Res. 49:1687-92.
Hotamisligil, G. S., D. L. Murray, L. N. Choy and B. M. Spiegelman (1994) “Tumor necrosis factor alpha inhibits signaling from the insulin receptor” Proc Natl Acad Sci USA 91:4854-8.
Inoguchi, T., Li, P., Umeda, F., Yu, H. Y., Kakimoto, M., Imamura, M., Aoki, T., Etoh, T., Hashimoto, T., Naruse, M., Sano, H., Utsumi, H., Nawata, H. (2000) “High glucose level and free fatty acid stimulate reactive oxygen species production through protein kinase C--dependent activation of NAD(P)H oxidase in cultured vascular cells” Diabetes. 49:1939-1945.
Khan, N., Sultana, S. (2004) “Induction of renal oxidative stress and cell proliferation response by ferric nitrilotriacetate (Fe-NTA): diminution by soy isoflavones” Chem Biol Interact. 149:23-35.
Koren, R., Hadari-Naor, I., Zuck, E., Rotem, C., Liberman, U. A., Ravid, A. (2001) “Vitamin D is a prooxidant in breast cancer cells” Cancer Res. 61:1439-44.
Korshunov, S. S., Skulachev, V. P., Starkov, A. A. (1997) “High protonic potential actuates a mechanism of production of reactive oxygen species in mitochondria” FEBS Lett. 416: 15-18.
Kouzarides, T., Ziff, E. (1989) “Leucine zippers of fos, jun and GCN4 dictate dimerization specificity and thereby control DNA binding” Nature. 340:568-71.
Lee, Y. H., Pratley, R. E. (2005) “The evolving role of inflammation in obesity and the metabolic syndrome” Curr Diab Rep. 5:70-75.
Li, Y., Bujo, H., Takahashi, K., Shibasaki, M., Zhu, Y., Yoshida, Y., Otsuka, Y., Hashimoto, N., Saito, Y. (2003) “Visceral fat: higher responsiveness of fat mass and gene expression to calorie restriction than subcutaneous fat” Exp Biol Med (Maywood) 228:1118-1123.
Lin, J. K. (2004) “Suppression of protein kinase C and nuclear oncogene expression as possible action mechanisms of cancer chemoprevention by Curcumin” Arch Pharm Res. 27:683-92.
Lin, T. K., Chen, S. D., Wang, P. W., Wei, Y. H., Lee, C. F., Chen, T. L., Chuang, Y. C., Tan, T. Y., Chang, K. C., Liou, C. W. (2005) “Increased Oxidative Damage with Altered Antioxidative Status in Type 2 Diabetic Patients Harboring the 16189 T to C Variant of Mitochondrial DNA” Ann N Y Acad Sci. 1042:64-69.
Mahadev, K., Motoshima, H., Wu, X., Ruddy, J. M., Arnold, R. S., Cheng, G., Lambeth, J. D., Goldstein, B. J. (2004) “The NAD(P)H oxidase homolog Nox4 modulates insulin-stimulated generation of H₂O₂ and plays an integral role in insulin signal transduction” Mol Cell Biol. 24:1844-54.
Manea, A., Constantinescu, E., Popov, D., Raicu, M. (2004) “Changes in oxidative balance in rat pericytes exposed to diabetic conditions” J Cell Mol Med. 8:117-126.
Melton, D. A. et al. (1984) “Efficient in vitro synthesis of biologically active RNA and RNA hybridization probes form plasmids containing a bacteriophage SP6 promoter” Nuc. Acids Res. 12:7035-7056.
Morris, K. L., Zemel, M. B. (2005) “1,25-dihydroxyvitamin D3 modulation of adipocyte glucocorticoid function” Obes Res. 13:670-677.
Miwa, S., Brand, M. D. (2003) “Mitochondrial matrix reactive oxygen species production is very sensitive to mild uncoupling” Biochem Soc Trans. 31:1300-1.
Nomura, M., Ma, W., Chen, N., Bode, A. M., Dong, Z. (2000) “Inhibition of 12-O-tetradecanoylphorbol-13-acetate-induced NF-kappaB activation by tea polyphenols, (−)-epigallocatechin gallate and theaflavins” Carcinogenesis. 21:1885-90.
Ofei, F., S. Hurel, J. Newkirk, M. Sopwith and R. Taylor (1996) “Effects of an engineered human anti-TNF-alpha antibody (CDP571) on insulin sensitivity and glycemic control in patients with NIDDM” Diabetes 45:881-885.
Quinn, L. S., Strait-Bodey, L., Anderson, B. G., Argiles, J. M., Havel, P. J. (2005) “Interleukin-15 stimulates adiponectin secretion by 3T3-L1 adipocytes: Evidence for a skeletal muscle-to-fat signaling pathway” Cell Biol Int. 29:449-57
Reeves, P. G. (1997) “Components of the AIN-93 diets as improvements in the AIN-76A diet” J Nutr. 127:838S-841S.
Schulze-Osthoff, K., Bauer, M. K., Vogt, M., Wesselborg, S. (1997) “Oxidative stress and signal transduction” Int J Vitam Nutr Res. 67:336-42.
Shangari, N., O'Brien, P. J. (2004) “The cytotoxic mechanism of glyoxal involves oxidative stress” Biochem Pharmacol. 68:1433-1442.
Shi, H., Norman, A. W., Okamura, W. H., Sen, A., Zemel, M. B. (2001) “1alpha,25-Dihydroxyvitamin D3 modulates human adipocyte metabolism via nongenomic action” FASEB J. 15:2751-2753.
Shi, H., Norman, A. W., Okamura, W. H., Sen, A., Zemel, M. B. (2002) “1α,25-dihydroxyvitamin D₃ inhibits uncoupling protein 2 expression in human adipocytes” FASEB J. 16:1808-1810.
Simeone, A. M., Tari, A. M. (2004) “How retinoids regulate breast cancer cell proliferation and apoptosis” Cell Mol Life Sci. 61:1475-84.
Soares, A. F., Guichardant, M., Cozzone, D., Bemoud-Hubac, N., Bouzaidi-Tiali, N., Lagarde, M., Geloen, A. (2005) “Effects of oxidative stress on adiponectin secretion and lactate production in 3T3-L1 adipocytes” Free Radic Biol Med. 38:882-889.
Song, Y. S., Rosenfeld, M. E. (2004) “Methionine-induced hyperhomocysteinemia promotes superoxide anion generation and NFkappaB activation in peritoneal macrophages of C57BL/6 mice” J Med Food. 7:229-34.
Sonta, T., Inoguchi, T., Tsubouchi, H., Sekiguchi, N., Kobayashi, K., Matsumoto, S., Utsumi, H., Nawata, H. (2004) “Evidence for contribution of vascular NAD(P)H oxidase to increased oxidative stress in animal models of diabetes and obesity” Free Radic Biol Med 37:115-123.
Sorescu, D., Weiss, D., Lassegue, B., Clempus, R. E., Szocs, K., Sorescu, G. P., Valppu, L., Quinn, M. T., Lambeth, J. D., Vega, J. D., Taylor, W. R., Griendling, K. K. (2002) “Superoxide production and expression of nox family proteins in human atherosclerosis” Circulation 105:1429-1435.
Sun, X. C., Morris, K., Zemel, M. B. (2005) “1, 25(OH)2D3 and reactive oxygen species interatively stimulate angiotensinogen expression in differentiated 3T3-L1 adipocytes” FASEB J. 19:A70 (abstract).
Sun, X. C., Zemel, M. B. (2004a) “Calcium and Dairy Products Inhibit Weight and Fat Regain during Ad Libitum Consumption Following Energy Restriction in Ap2-Agouti Transgenic Mice” J Nutr. 134:3054-3060.
Sun, X., Zemel, M. B. (2004b) “Role of uncoupling protein 2 (UCP2) expression and 1α, 25-dihydroxyvitamin D₃ in modulating adipocyte apoptosis” FASEB J. 18:1430-1432.
Sun, X. C., Zemel, M. B. (2004c) “Dual effect of 1-alpha,25-dihydroxyvitamin D-3 on adipocyte apoptosis” FASEB J. 18:A49 (abstract).
Sun, X. C., Zemel, M. B. (2004d) “Reactive oxygen species stimulate cell proliferation and down-regulate UCP2 expression in 3T3-L1 adipocytes” Obesity Research 11:A37(abstr).
Sun, X. C., Zemel, M. B (2006) “Dietary calcium regulates ROS production in aP2-agouti transgenic mice on high fat/high sucrose diets” Int J Obesity, in press.
Suzuki, K., Ito, Y., Ochiai, J., Kusuhara, Y., Hashimoto, S., Tokudome, S., Kojima, M., Wakai, K., Toyoshima, H., Tamakoshi, K., Watanabe, Y., Hayakawa, N., Maruta, M., Watanabe, M., Kato, K., Ohta, Y., Tamakoshi, A.; JACC Study Group (2003) “Relationship between obesity and serum markers of oxidative stress and inflammation in Japanese” Asian Pac J Cancer Prev. 4:259-266.
Thompson, W. G., Rostad Holdman, N., Janzow, D. J., Slezak, J. M., Morris, K. L., Zemel, M. B. (2005) “Effect of energy-reduced diets high in dairy products and fiber on weight loss in obese adults” Obes Res. 2005:13:1344-53.
Valle, M., Martos, R., Gascon, F., Canete, R., Zafra, M. A., Morales, R. (2005) “Low-grade systemic inflammation, hypoadiponectinemia and a high concentration of leptin are present in very young obese children, and correlate with metabolic syndrome” Diabetes Metab. 31 :55-62.
Volk, T., Hensel, M., Kox, W. J. (1997) “Transient Ca²⁺changes in endothelial cells induced by low doses of reactive oxygen species: role of hydrogen peroxide” Mol Cell Biochem. 171:11-21.
Upham, B. L., Kang, K. S., Cho, H. Y., Trosko, J. E. (1997) “Hydrogen peroxide inhibits gap junctional intercellular communication in glutathione sufficient but not glutathione deficient cells” Carcinogenesis 18:37-42.
Xu, H., Barnes, G. T., Yang, Q., Tan, G., Yang, D., Chou, C. J., Sole, J., Nichols, A., Ross, J. S., Tartaglia, L. A., Chen, H. (2003) “Chronic inflammation in fat plays a crucial role in the development of obesity-related insulin resistance” J Clin Invest. 112:1821-1830.
Xue, B., Zemel, M. B. (2000) “Relationship between human adipose tissue agouti and fatty acid synthase (FAS)” J Nutr. 130:2478-2481.
Xue, B., Moustaid, N., Wilkison, W. O., Zemel, M. B. (1998) “The agouti gene product inhibits lipolysis in human adipocytes via a Ca²⁺-dependent mechanism” FASEB J. 12:1391-1396.
Wajchenberg, B. L. (2000) “Subcutaneous and visceral adipose tissue: their relation to the metabolic syndrome” Endocr Rev. 21:697-738.
Weisberg, S. P., McCann, D., Desai, M., Rosenbaum, M., Leibel, R. L., Ferrante, A. W. Jr. (2003) “Obesity is associated with macrophage accumulation in adipose tissue” J Clin Invest. 112:1796-808.
Weitzman, S. A., Turk, P. W., Milkowski, D. H., Kozlowski, K. (1994) “Free radical adducts induce alterations in DNA cytosine methylation” Proc Natl Acad Sci USA. 91:1261 -4.
Zemel, M. B., Shi, H., Greer, B., Dirienzo, D., Zemel, P. C. (2000) “Regulation of adiposity by dietary calcium” FASEB J. 14:1132-1138.
Zemel, M. B. (2003) “Effects of mitochondrial uncoupling on adipocyte intracellular Ca2+ and lipid metabolism” J Nutr Biochem. 14:219-226.
Zemel, M. B. (2004) “Role of calcium and dairy products in energy partitioning and weight management” Am J Clin Nutr. 79:907S-912S.
Zemel, M. B., Thompson, W., Milstead, A., Morris, K., Campbell P. (2004) “Calcium and dairy acceleration of weight and fat loss during energy restriction in obese adults” Obes Res. 12:582-90.
Zemel, M. B. (2005a) “Calcium and dairy modulation of obesity risk” Obes Res. 13(1):192-193.
Zemel, M. B. (2005b) “The role of dairy foods in weight meanagement” J. Am Coll Nutr. 24(6 Suppl):537S-546S.
Zemel, M. B., Richards, J., Mathis, S., Milstead, A., Gebhardt, L., Silva, E. (2005a) “Dairy augmentation of total and central fat loss in obese subjects” Int J Obes Relat Metab Disord. 29:391-397.
Zemel, M. B., Richards, J., Milstead, A., Campbell, P. (2005b) “Effects of calcium and dairy on body composition and weight loss in African-American adults” Obes Res. 2005:13:1218-25.

We claim:

1. An in vitro method of screening compounds or compositions suitable for reducing the production of reactive oxygen species (ROS) comprising:
a) contacting one or more cell(s) with a composition comprising dietary material containing dietary calcium; and
b) measuring one or more of the following parameters:
i) intracellular concentrations of calcium in said one or more cell(s), wherein a decrease of intracellular calcium concentration in said cell(s) is indicative of a compound or composition suitable for use in reducing the production of ROS;
ii) UCP2 expression in said one or more cell(s), wherein an increase in UCP2 expression in said cell(s) is indicative of a compound or composition suitable for use in reducing the production of ROS;
iii) NADPH oxidase expression in said one or more cell(s), wherein a decrease in NADPH oxidase expression in said cell(s) is indicative of a compound or composition suitable for use in reducing the production of ROS;
iv) UCP3 expression in said one or more cell(s), wherein an increase in UCP3 expression in said cell(s) is indicative of a compound or composition suitable for use in reducing the production of ROS;
v) NADPH oxidase expression in said one or more cell(s), wherein a decrease in NADPH oxidase expression in said cell(s) is indicative of a compound or composition suitable for use in reducing the production of ROS;
vi) 11β-HSD expression in said one or more cell(s), wherein a decrease in the expression of 11β-HSD in said cell(s) is indicative of a compound or composition suitable for use in reducing the production of ROS;
vii) TNF-α, CD14, MIF, M-CSF, MIP, MCP-1, G-CSF or IL-6 expression in said one or more cell(s), wherein a decrease in the expression of TNF-α, CD14, MIF (macrophage inhibitory factor), MIP (macrophage inhibitory protein), M-CSF (macrophage colony stimulating factor), MCP-1 (monocyte chemoattractant protein-1), G-CSF (granulocyte colony stimulating factor) or IL-6 in said cell(s) is indicative of a compound or composition suitable for use in reducing the production of ROS; or
viii) IL-15 or adiponectin expression in said one or more cell(s), wherein an increase in the expression of IL-15 or adiponectin in said cell(s) is indicative of a compound or composition suitable for use in reducing the production of ROS.

2. The method according to claim 1, wherein said one or more cell(s) is a adipocyte or an adipocyte cell line.

3. The method according to claim 2, wherein said adipocyte or adipocyte cell line is human(s) or a murine.

4. A method of identifying or screening compounds or compositions suitable for reducing the production of reactive oxygen species (ROS) comprising:
a) orally administering a composition comprising dietary material containing dietary calcium; and
b) measuring one or more of the following parameters:
i) intracellular calcium concentrations in cells of said at least one test subject and at least one control subject, wherein a decrease of intracellular calcium concentration in the cells of a test subject as compared to the intracellular concentrations of calcium in the cells of at least one control subject is indicative of a compound, composition, combination of compounds or combination of compositions suitable for use in reducing the production of ROS in a subject;
ii) UCP2 expression in cells of said at least one test subject and at least one control subject, wherein an increase of UCP2 expression in the cells of a test subject as compared to the UCP2 expression in the cells of at least one control subject is indicative of a compound, composition, combination of compounds or combination of compositions suitable for use in reducing the production of ROS in a subject;
iii) NADPH oxidase expression in cells of said at least one test subject and at least one control subject, wherein a decrease of NADPH oxidase expression in the cells of a test subject as compared to the NADPH oxidase expression in the cells of at least one control subject is indicative of a compound, composition, combination of compounds or combination of compositions suitable for use in reducing the production of ROS in a subject;
iv) UCP3 expression in skeletal muscle cells of said at least one test subject and at least one control subject, wherein an increase in UCP3 expression in the skeletal muscle cells of a test subject as compared to UCP3 expression in the skeletal muscle cells of at least one control subject is indicative of a compound, composition, combination of compounds or combination of compositions suitable for use in reducing the production of ROS in a subject;
v) NADPH oxidase expression in skeletal muscle cells of said at least one. test subject and at least one control subject, wherein a decrease of NADPH oxidase expression in the skeletal muscle cells of a test subject as compared to the NADPH oxidase expression in the skeletal muscle cells of at least one control subject is indicative of a compound, composition, combination of compounds or combination of compositions suitable for use in reducing the production of ROS in a subject;
vi) 11β-HSD expression in visceral adipocyte tissue or cells of said at least one test subject and at least one control subject, wherein a decrease of 11β-HSD expression in the visceral adipocyte tissue or cells of a test subject as compared to the 11β-HSD expression in the visceral adipocyte tissue or cells of at least one control subject is indicative of a compound, composition, combination of compounds or combination of compositions suitable for use in reducing the production of ROS in a subject;
vii) TNF-α, CD14, MIF, MIP, M-CSF, MCP-1, G-CSF or IL-6 expression in said one or more cell(s), wherein a decrease in the expression of TNF-α, CD14, MIF, MIP, M-CSF, MCP-1, G-CSF or IL-6 in said cell(s) is indicative of a compound or composition suitable for use in reducing the production of ROS; or
viii) IL-15 or adiponectin expression in said one or more cell(s), wherein an increase in the expression of IL-15 or adiponectin in said cell(s) is indicative of a compound or composition suitable for use in reducing the production of ROS.

5. The method according to claim 4, wherein a candidate compound, combination of candidate compounds, candidate composition, or combination of candidate compositions is administered to at least one test subject orally as a component of the diet of said test subject or as dietary calcium to said test subject.

6. A method of treating diseases associated with reactive oxygen species (ROS) comprising the oral administration of dietary calcium or dietary material containing dietary calcium to an individual in need of such treatment in amounts sufficient to decrease the intracellular concentrations of calcium in the cells of the individual.

7. The method according to claim 6, wherein said method further comprises a step of diagnosing or identifying an individual as having a disease or disorder associated with ROS or suffering from elevated ROS levels.

8. A method of altering the expression of cytokines in an individual or the cytokine profile of an individual comprising the oral administration of dietary calcium or dietary material containing dietary calcium that decrease intracellular calcium levels to an individual in need of such treatment in amounts sufficient to decrease intracellular levels of calcium in the cells of the individual, decrease TNF-α, CD14, MIP, MIF, M-CSF, MCP-1, G-CSF or IL-6 expression and increase the expression of IL-15, adiponectin, or both IL-15 or adiponectin in the individual.
